(19) United States
(12) Patent Application Publication
(10) Pub. No.: US 2014/0205669 A1
(43) Pub. Date: Jul. 24, 2014

(54) dhS1P AND USE OF SAME AS AN ANTICANCER THERAPEUTIC AND IMMUNOMODULATOR

(71) Applicant: THE PENN STATE RESEARCH FOUNDATION, University Park, PA (US)

(72) Inventors: Brian M. Barth, Hummelstown, PA (US); Mark Kester, Harrisburg, PA (US); James H. Adair, State College, PA (US); Todd E. Fox, Hershey, PA (US)

(73) Assignee: THE PENN STATE RESEARCH FOUNDATION, University Park, PA (US)

(21) Appl. No.: 14/090,764

(22) Filed: Nov. 26, 2013

Related U.S. Application Data
(60) Provisional application No. 61/731,081, filed on Nov. 29, 2012.

(51) Int. Cl. A61K 31/661; A61K 35/00; A61K 45/06

(52) U.S. Cl. CPC A61K 31/661; A61K 45/06; A61K 35/13
USPC 424/490; 514/114; 424/93.7

(57) ABSTRACT

Use of dhS1P and/or PhotoImmunoNanoTherapy as a therapeutic agent is described. Administration of therapeutically effective amounts of dhS1P decrease the number of Myeloid Derived Suppressor Cells and immune suppression in cancer patients. Administration of therapeutically effective amounts of dhS1P can be used as an adjuvant to conventional cancer therapies including immunotherapies. Therapeutic results can be achieved by directly administering dhS1P and/or by indirectly increasing the amount of dhS1P at the tumor site. The therapy permits the patient's immune system to recognize and eliminate cancer cells reducing tumor size and extending patient survival.

### CROSS-REFERENCE TO RELATED APPLICATIONS

This application claims priority under 35 U.S.C. §119, and is related to, U.S. Provisional Application Ser. No. 61/731,081 filed on Nov. 29, 2012 and entitled Immunomodulatory Properties of dhS1P as a Standalone and/or Adjuvant Anticancer Therapeutic. The entire contents of this patent application are hereby expressly incorporated herein by reference including, without limitation, the specification, claims, and abstract, as well as any figures, tables, or drawings thereof.

### GRANT REFERENCE

This invention was made with government support under NIH Grant NIH grant R01—CA117926 awarded by the National Institutes of Health. The government has certain rights in the invention.

### BACKGROUND OF THE INVENTION

The development of more efficacious and less toxic cancer therapies is a priority due to the prevalence and poor prognosis of the disease. Current cancer therapies are highly toxic and offer a range of potential efficacy that varies with the subtype and staging of the disease. Photodynamic therapy (PDT) has emerged as an alternative to traditional chemotherapy and radiation therapy for the treatment of certain types of cancer, but not breast or pancreatic cancer or metastatic osteosarcoma. PDT takes advantage of an appropriate wavelength of light exciting a photosensitizer to an excited triplet energy state. In the presence of molecular oxygen, which resides in a ground triplet state, energy is transferred to relax the excited state of the photosensitizer. This energy transfer in turn excites molecular oxygen to form excited singlet state oxygen (¹O₂). The effects of PDT have been attributed to ¹O₂ triggering cell death via damaging oxidation or redox-sensitive cellular signaling pathways. Unfortunately PDT suffers from disadvantages associated with photosensitizer toxicity, a lack of efficacious and selective photosensitizers, as well as an inability of light to sufficiently penetrate through tissues to reach tumors deep within the body. The efficacy of conventional PDT is limited by photosensitizers that offer limited optical characteristics and high toxicity. For these reasons, PDT is currently limited primarily to the treatment of cancers of the skin and esophagus.

Recently the synthesis and utility of calcium phosphosilicate nanoparticles (CPSNPs) was described. Biocompatible CPSNPs were shown to increase the quantum efficiency and photostability of encapsulated fluorescent dyes. Furthermore, surface functionalization with polyethylene glycol (PEG) allowed for efficient in vivo imaging using indocyanine green (ICG)-loaded CPSNPs via enhanced permeation and retention of the particles within xenografted breast and pancreatic cancer tumors. ICG is a near-infrared (NIR) fluorescing dye that has been approved by the Food and Drug Administration of the United States of America for use in medical imaging. The utility of ICG encapsulated within CPSNPs for deep tissue imaging is related to the ability of longer wavelength NIR light to penetrate through tissue. Surface targeting moieties were successfully coupled to CPSNPs, which allowed for specific targeting to breast or pancreatic cancer tumors to improve diagnostic imaging.

Immunosuppression is a major obstacle to effective treatment of cancer and can be a contributing factor to therapy resistance. Recently, immune-suppressive cells have gained notoriety as critical cellular regulators by which tumors evade immunity and overcome therapeutic intervention. These suppressive cells include a heterogeneous population of immature myeloid cells expanded systemically as a consequence of a profound tumor-associated pro-inflammatory milieu, likely prematurely mobilized myeloid progenitors, and which have also been referred to as myeloid-derived suppressor cells (MDSCs). MDSCs typically bear the expression of multiple cell-surface markers that are normally specific for monocytes, macrophages or DCs and are comprised of a mixture of myeloid cells with granulocytic and monocytic morphology. Normal bone marrow contains 20-30% of IMCs, and IMCs make up small proportion (2-4%) of spleen cells. IMCs/MDSCs are not typically found in lymph nodes in mice. In humans, for healthy individuals, IMCs comprise ˜0.5% of peripheral blood mononuclear cells. In the case of cancer, IMCs specifically expanded and mobilized by tumor-associated factors exert an immunosuppressive phenotype that distinguishes them as MDSCs. Anticancer T-cell-dependent and -independent immune responses have been shown to be negatively regulated by MDSCs in a diversity of models of cancer. In addition to tumors, MDSCs are found at high numbers in the peripheral circulation and in organs such as the spleen and liver, and their systemic numbers are directly correlated with tumor burden. These immunosuppressive myeloid cells have been identified in both humans and mice, including athymic nude mice, with populations defined by the presence of particular combinations of surface antigens. In mice, MDSCs are Gr-1+CD11b+ granulocytic or monocytic cells, while in humans they are primarily defined within a CD14-HLA-DR-CD33+CD11b+ population. MDSCs can be identified by intrinsic features of NADPH oxidase activity, arginase activity, and/or nitric oxide synthase. Alternatively, MDSCs in mice can be identified by a Gr-1+ and/or CD11b+ phenotype. Because human cells do not express a marker homologous to mouse Gr1, they are typically phenotypically identified by a Lin⁻HLA⁻DR⁻CD33⁺ and/or CD11b⁺CD14⁻CD33⁺ phenotype. In tumor tissues, MDSCs can be differentiated from tumor-associated macrophages (TAMs) by their high expression of Gr-1 (not expressed by TAMs) by their low expression of F4/80 (expressed by TAMs), by the fact that a large proportion of MDSCs have a granulocytic morphology and based the upregulated expression of both arginase and inducible nitric oxide synthase by MDSCs but not TAMs.

MDSCs represent an intrinsic part of the myeloid-cell lineage and are a heterogeneous population that is comprised of myeloid-cell progenitors and precursors of myeloid cells. Typically, the immature myeloid cells (IMCs) rapidly differentiate into mature granulocytes, macrophages or dendritic cells (DCs). However, in pathological conditions, such as cancer, a partial block in the differentiation of IMCs into mature myeloid cells results in an expansion of the population of IMCs. These cells, particularly in a pathological context, results in the upregulated expression of immune suppressive factors. Examples of such factors include arginase, NO (nitric oxide) and reactive oxygen species (ROS). Ultimately, this results in the expansion of an IMC population that has immune suppressive activity called MDSCs. MDSCs are considered a major contributor to the immune dysfunction of most patients with sizeable tumor burdens. While attempting to determine the underlying basis for ICG-CPSNP PDT's robust antitumor effect described above, the inventors turned to investigation of MDSCs.

Approximately twenty years ago, researchers first identified hematopoietic suppressor cells which were then called “natural suppressor” cells. Approximately ten years later, after observing large numbers of these cells in the blood of cancer patients and mice with tumors, researchers were able to determine that the cells were derived from myeloid tissues as well as their role in suppressing immune function in tumors. To date, MDSCs have been documented in most patients and mice with cancer, where their production is encouraged by various factors produced by tumor cells and host cells in the tumor environment.

MDSC levels are driven by tumor burden and the diversity of factors produced by the tumor and host cells. MDSCs directly interfere with T cell mediated immunity, and dendritic and natural killer cell function which, in turn, reduces the ability for a patient's immune system to attack cancer cells. Therefore significant effort is underway toward the development of therapies that decrease MDSCs.

The inventors have discovered that isolated MDSCs are decreased by treatment with dihydrosphingosine-1-phosphate (dhS1P), but not sphingosine-1-phosphate (S1P), while dhS1P induced a concomitant expansion of antitumor lymphocytes bearing the surface characteristics of B cells. Adoptive transfer of these dhS1P-induced B cells into tumor-bearing mice effectively blocked breast cancer tumor growth and extended the survival of mice with orthotopic pancreatic cancer tumors. Effective therapeutic delivery of dhS1P using PhotoImmunoNanoTherapy was accomplished on multiple cancer models. Direct injections of dhS1P into pancreatic tumor-bearing mice also resulted in decreased tumor growth and improved life expectancy. These results demonstrate the use of dhS1P as a broad and effective therapeutic agent for cancer.

Sphingolipids represent a broad classification of lipids with important roles in membrane biology and signal transduction. The de novo synthesis of sphingolipids, and therefore the initial formation of the sphingoid backbone, begins with the condensation of the amino acid serine and the fatty acid palmitate to yield the intermediate 3-ketodihydrosphingosine (also known as 3-ketosphinganine). Enzymatic reduction results in the formation of dihydrosphingosine (sphinganine), which serves as the precursor for dhS1P or for dihydroceramide and subsequently ceramide. It is at this point in the de novo synthetic pathway at which an initial role for dhS1P was considered, namely as an alternative metabolic pathway to prevent the ultimate synthesis of the pro-apoptotic sphingolipid ceramide. The generation of dhS1P is catalyzed by sphingosine kinase, the same enzyme that catalyzes the formation of sphingosine-1-phosphate (S1P). Although sphingosine kinase can phosphorylate either sphingosine or dihydrosphingosine, the cellular location of the enzyme, and therefore more profound access to certain substrates, was suggested to determine whether S1P or dhS1P would be preferentially produced. S1P is a catabolic product of ceramide, generated via deacylation to yield sphingosine and then subsequent phosphorylation, and as such has gained considerable attention for its biological roles that oppose those of ceramide. On the other hand, dhS1P has mostly been ignored largely so because the mass levels of dhS1P are often an order of magnitude less than S1P. Furthermore, most researchers have assumed that dhS1P and S1P share identical biological roles due to an almost identical structural similarity that only differs by the presence of a 4-5 trans double bond in SIP.

As opposed to the pro-apoptotic and pro-cellular stress sphingolipid ceramide, S1P has been largely characterized as being pro-survival, and pro-mitogenic, as well as playing profound roles in development and immune modulation. Specific G protein-coupled receptors have been identified for S1P, and most of the biological roles of the lipid have been traced to these receptors. In addition, S1P has also recently been shown to interact with targets in the nucleus and modulate the cellular epigenetic program. The elevation of S1P mass and an increase in the abundance and activity of sphingosine kinase has been well-documented in cancer. In contrast, research has largely shown that the pro-apoptotic sphingolipid ceramide is diminished in cancer but that a diversity of chemotherapeutics as well as radiation therapy can increase its levels. Furthermore, extensive research has focused on the development of inhibitors of sphingosine kinase as anticancer therapeutics. While these efforts revolve around the well-excepted role of S1P in cancer, they fail to address any role for dhS1P due to its structural similarities to S1P and relatively low mass levels.

In addition to having documented roles in the pathogenesis of cancer, S1P has also been extensively shown to modulate the immune system. The trafficking of immune cells in response to a gradient of SIP, and activation of immune effectors, are considered to be primary immunomodulatory roles for S1P. Trafficking of thymic progenitors to the thymus, the egress of progenitors out of the bone marrow, and the homing of immune effectors, all have been directly attributed to S1P exerting its influence via specific G protein-coupled receptors. As such, specific agonists and antagonists of these receptors have gained attention as potent immunomodulatory agents for therapeutic use following transplant, as agents to counteract severe autoimmune disorders, and for the utility of treating severe allergy. A recent study has shown that the S1P analog FTY720 can reduce immunosuppression by regulatory T cells by modulating the S1P₁ receptor. Unfortunately, the precise role of this analog is debatable as it can both elicit S1P₁-mediated signaling by acting like S1P as well as block S1P-signaling by inducing internalization of the receptor. There are no specifically defined roles for S1P, or dhS1P, in the regulation of myeloid-derived suppressor cells (MDSCs), as well as none for the development of antitumor immune effectors. As in the case of cancer, no specific immunomodulatory roles have been ascribed to dhS1P as most research focuses on the structurally-related and more abundant S1P. In addition, some concern exists over the development of S1P-based immunomodulatory agents as these could behave differentially in the context of S1P and cancer biology evidenced in part by a study showing that targeted disruption of the S1P₂ receptor resulted in the development of large diffuse B-cell lymphomas.

More recently, some studies have begun to shed light on specific biochemical roles for dhS1P. These studies have occurred in the context of the profibrotic disease scleroderma and have focused on the transforming growth factor beta (TGFβ) signaling pathway and the tumor suppressor PTEN. In scleroderma, and other fibrotic diseases, excessive production of components of the extracellular matrix (ECM) occurs, and this has been linked to TGFβ and S1P-signaling. Initially, studies showed that dhS1P could exert a differential effect by activating the NF-κB signaling pathway and by inducing matrix metalloproteinase (MMP) 1 activity to degrade the ECM. Further studies showed that dhS1P could potentiate the C-terminal phosphorylation of PTEN which resulted in its nuclear translocation and subsequent interference with downstream biochemical effectors of the TGFβ pro-fibrotic signaling pathway. These studies provided the first distinct role for dhS1P at the biochemical level, but did so in a context that has confusing implications in the context of cancer biology. First, the activation of TGFβ signaling has been attributed both pro-inflammatory and anti-inflammatory roles. Second, the NF-κB transcription factor is almost exclusively associated with the production of pro-inflammatory mediators. Of particular concern, a profound pro-inflammatory milieu is well associated with the development of immunosuppression and cancer, and in particular to the development of MDSCs. Third, the activation of MMPs and the subsequent degradation of the ECM are classically associated with cancer invasion and metastasis. Lastly, the translocation of PTEN to the nucleus removes this tumor suppressor from the cellular location needed to exert influence as a tumor suppressor, potentially augmenting the capacity of the Akt/PKB signaling cascade to exert a pro-oncogenic program. These points discourage the use of dhS1P to treat cancer. Additionally, in light of the commonly held assumption that dhS1P is just a cousin to the more abundant, and structurally related S1P, would lead one skilled in the art to conclude that dhS1P would not be effective in the treatment of cancer and depletion of immunosuppressive MDSCs.

It is a primary object, feature or advantage of the present invention to improve over the state of the art.

A further object, feature or advantage of the invention is to provide a novel method and/or composition for the treatment of tumors that greatly reduces toxic side effects to the patient compared to conventional cancer treatments.

A further object, feature or advantage of the invention is to provide a novel method and/or composition for reducing a patient's number of MDSCs.

A further object, feature or advantage of the invention is to provide a novel method and/or composition for the treatment of tumors that stimulates a patient's immune system.

A further object, feature or advantage of the invention is to provide a novel method and/or composition for cancer treatment that inhibits tumor growth.

A further object, feature or advantage of the invention is to provide a novel method and/or composition for cancer treatment that results in tumor reduction.

A further object, feature or advantage of the invention is to provide a novel method and/or composition for cancer treatment that is effective for treatment of various types of cancer.

A further object, feature or advantage of the invention is to provide a novel method and/or composition for cancer treatment that has little effect on the patient's healthy tissue.

A further object, feature or advantage of the invention is to provide a novel method and/or composition for cancer treatment that can be used prior to, concurrently with, or subsequent to other methods and/or compositions for treatment of tumors.

A further object, feature or advantage of the invention is to provide a novel method and/or composition for cancer treatment that increases the effectiveness of an additional tumor therapy administered as part of a treatment regimen compared to administration of the additional tumor therapy alone.

One or more of these and/or other objects, features, or advantages of the present invention will become apparent from the specification and claims that follow. It should be understood, however, that the following description and the specific examples, while indicating preferred embodiments of the invention, are given by way of illustration only. Various changes and modifications within the spirit and scope of the disclosed invention will become readily apparent to those skilled in the art from reading the following description and from reading the other parts of the present disclosure. No single embodiment of the invention need fulfill all of any of the objects stated herein.

### BRIEF SUMMARY OF THE INVENTION

The present invention relates to novel and previously unknown uses of dhS1P. The present invention provides for methods and compositions for the treatment of tumors. In another aspect, the present invention provides methods and compositions for the reduction of MDSCs. In another aspect, the present invention provides methods and compositions for the stimulation of a patient's immune system. In one aspect, the method includes administering an effective amount of dhS1P to a patient to treat tumors. Preferably, the tumor to be treated is characterized as having a high number of MDSCs. In another aspect, the dhS1P may be part of a treatment regimen including at least one additional tumor treatment therapy. Preferably, the additional therapy is an immunologic therapy. In another aspect, the method includes administering an effective amount of dhS1P to a patient to reduce the patient's number of MDSCs. In another aspect, the method includes administering an effective amount of dhS1P to a patient to stimulate the patient's immune system. In another aspect, the effective amount of dhS1P may be delivered in conjunction with or using PhotoImmunoNanoTherapy.

The invention also includes a pharmaceutical composition comprising dhS1P and a carrier. In certain embodiments, the shS1P pharmaceutical composition includes an encapsulated nanoparticle includes dhS1P.

### BRIEF DESCRIPTION OF THE DRAWINGS

FIG. 1 (A-F) shows PhotoImmunoNanoTherapy decreases tumor burden and improves survival while simultaneously diminishing the systemic inflammatory and myeloid immune milieus (ICG: indocyanine green, Ghost: empty, CPSNP: calcium phosphosilicate nanoparticles, COOH: citrate functionalized, PEG: PEGylated). (A) Tumor growth following PhotoImmunoNanoTherapy was monitored in athymic nude mice engrafted with human MDA-MB-231 breast cancer cells. ANOVA, *p<0.05 compared to all, n≧5. (B) Tumor growth following PhotoImmunoNanoTherapy was monitored in BALB/cJ mice engrafted with murine 410.4 breast cancer cells. ANOVA, *p<0.05 compared to all, n≧8. (C) Tumor growth following PhotoImmunoNanoTherapy was monitored in NOD.CB17-Prkdc^{scid}/J mice engrafted with murine 410.4 breast cancer cells. ANOVA, *p<0.001 compared to all, n≧7. (D) Tumor growth following PhotoImmunoNanoTherapy was monitored in C57BL/6J mice engrafted with murine Panc-02 pancreatic cancer cells. ANOVA, *p<0.05 compared to all, n≧6. (E) Survival of athymic nude mice orthotopically implanted with human BxPC-3-GFP pancreatic cancer cells was monitored following PhotoImmunoNanoTherapy. Logrank test, *p<0.05 compared to all, n=5. (F) Survival of athymic nude mice with experimental lung metastases of human SAOS-2-LM7 osteosarcoma cells was monitored following PhotoImmunoNanoTherapy. Logrank test, *p<0.05, n=5.

FIG. 2 (A-C) shows PhotoImmunoNanoTherapy diminishes the systemic inflammatory and myeloid immune milieus. (A-B) Splenic IMCs (immature myeloid cells) (Gr-1+CD11b+) were decreased five days following PhotoImmunoNanoTherapy of various cancer models (A) Representative dot plots from 410.4 breast tumor-bearing BALB/cJ mice. (B) Percent of immature myeloid cells determined by flow cytometry of splenocytes. ANOVA, *p<0.05 compared to all, #p=0.05 compared to all, n≧4. (C) Serum collected from tumor-bearing athymic nude mice one day following PhotoImmunoNanoTherapy was collected and a cytokine multiplex assay was used to quantify IL-1β, IL-6, IL-12(p70), IL-10, IFNγ, and TNFα were determined. ANOVA, *p<0.05 compared to all, n=3.

FIG. 3 (A-C) shows splenocytes harvested from MDA-MB-231 tumor-bearing athymic nude mice were harvested, and prepared for multicolor flow cytometry. (A) Initially, MDSC-like cells were gated as Gr-1+CD11b+. Respective gating evaluated the presence of CD44, CD115, and the gp91phox subunit of the NADPH oxidase. (B) Splenocytes were incubated with antibodies targeting CD11b, and the LY-6G and LY-6C subsets of Gr-1. (C) Splenocytes were incubated with 10 μM of the redox-sensitive indicator dicholorofluorescein (DCF) with or without 250 nM PMA for 30 minutes. DCF-fluorescence was evaluated within the Gr-1+CD11b+MDSC-like cell population.

FIG. 4 (A-D) shows flow cytometric analysis of splenic B cells from tumor-bearing mice following NIR treatment. (A-B) Splenic B cells (A; Gr-1−CD11b−CD19+CD45R B220+), and NK cells (B; CD49b DX5+) evaluated from MDA-MB-231 tumor-bearing athymic nude mice sacrificed 5 days following NIR-treatment. Mice received either PBS, PEGylated empty (ghost)-CPSNPs, or PEGylated ICG-loaded CPSNPs, 24 hours prior to NIR treatment. **p<0.01, ^{#}p=3. ^{#}p<0.001, n=4. (C-D) Splenic B cells (C; Gr-1−CD11b−CD19+CD45R B220+) (**p<0.001, n=4), and NK cells (D; CD49b DX5+) (^{#}p≦0.01, n≧3), evaluated from 410.4 tumor-bearing BALB/cJ mice sacrificed 5 days following NIR-treatment. Mice received either PBS, PEGylated empty (ghost)-CPSNPs, or PEGylated ICG-loaded CPSNPs, 24 hours prior to NIR treatment.

FIG. 5 (A-H) shows PhotoImmunoNanoTherapy increases the serum levels of phosphorylated bioactive sphingolipids. MDA-MB-231 breast tumor-bearing athymic nude mice, or 410.4 breast tumor-bearing BALB/cJ mice, received PBS, empty (ghost) CPSNPs, or ICG-loaded (PEGylated) CPSNPs, followed 24 hours later by NIR treatment. Tumors were collected and prepared 5 days following NIR treatment, lipids were extracted, and LC-MS³ was used to analyze levels of (A-B) ceramide species (ANOVA, #p<0.05 compared to Ghost-CPSNP-PEG, n≧3), (C) sphingosine (ANOVA, *p<0.05 compared to all), (D) sphingosine-1-phosphate (SIP) (ANOVA, *p<0.05 compared to all, #p<0.05 compared to Ghost-CPSNP-PEG, n≧3), (E) dihydrosphingosine, and (F) dihydrosphingosine-1-phosphate (dhS1P). (G) SIP (ANOVA, *p<0.05 compared to all), and (H) dhS1P (ANOVA, *p<0.05 compared to all, unpaired student's t-test, #p<0.05 compared to Ghost-CPSNP-PEG only, n≧3), were quantified by LC-MS³ in the serum of human MDA-MB-231 subcutaneous breast cancer tumor-bearing athymic nude mice, murine 410.4 subcutaneous breast cancer tumor-bearing BALB/cJ mice, human BxPC-3-GFP orthotopic pancreatic cancer tumor-bearing athymic nude mice, and human SAOS-2-LM7 experimental lung-metastatic osteosarcoma tumor-bearing athymic nude mice five days following treatment with PhotoImmunoNanoTherapy.

FIG. 6 (A-C) shows the therapeutic efficacy of PhotoImmunoNanoTherapy requires sphingosine kinase 2. (A) Experimental model wherein cancer cells treated in culture with PhotoImmunoNanoTherapy, are harvested, and then injected systemically into tumor-bearing mice. The premise was that treatment would trigger the release of sphingosine-1-phosphate (S1P) and dihydrosphingosine-1-phosphate (dhS1P) and that one of these or both would exert an antitumor effect. This strategy allowed interference with S1P/dhS1P-producing sphingosine kinase (SphK) with siRNA in the cultured cancer cells. (B) Cultured MDA-MB-231 cells, first exposed to siRNA (siSCR: scrambled control siRNA, siSK1: SphK1 siRNA, siSK2: SphK2 siRNA), were treated in culture with PhotoImmunoNanoTherapy and then injected into MDA-MB-231 tumor-bearing athymic nude mice. Alternatively, MDA-MB-231 cells exposed only to scrambled control siRNA without any near-infrared (NIR) light treatment were injected as controls. ANOVA, *p<0.05 compared to all, #p<0.05 compared to PBS, untreated cells exposed to scrambled control siRNA, $p<0.05 compared to NIR-treated cells exposed to SphK1 siRNA, n≧5. (C) 410.4 cells stably expressing either SphK1 or SphK2 were exposed to normally non-toxic PhotoImmunoNanoTherapy conditions and cellular viability was evaluated. ANOVA, *p<0.05 compared to all, n=4.

FIG. 7 (A-C) shows isolated immature myeloid cells (IMCs) from tumor-bearing athymic nude mice are decreased by dhS1P treatment while cells with B-cell characteristics are expanded from hematopoietic progenitors. (A) Splenic IMCs (Gr-1+CD11b+, also defined as MDSCs: myeloid-derived suppressor cells) were isolated from MDA-MB-231 tumor-bearing athymic nude mice and exposed to BSA, S1P (5 μM), or dhS1P (5 μM). Following 24-hour culture incubation, cells were labeled with specific antibodies and analyzed by multicolor flow cytometry (red: IMCs; blue: possible B-cells). (B) Splenic IMCs were isolated from MDA-MB-231 tumor-bearing athymic nude mice and cultured (5×104 cells/mL) in GEMM-supportive semi-solid media with BSA, SIP (5 μM), or dhS1P (5 μM). GEMM colonies (multipotent myeloid progenitor cells) were visualized and counted after 3 weeks of culture. ANOVA, *p<0.01 compared to no treatment or BSA-treatment, ***p<0.001 compared to S1P-treatment, n≧3. (C) Splenic hematopoietic progenitors (Lineage-Sca-1+CD117+) were isolated from MDA-MB-231 tumor-bearing athymic nude mice and exposed to BSA, SIP (5 μM), or dhS1P (5 μM). Following 24-hour culture incubation, cells were labeled with specific antibodies and analyzed by multicolor flow cytometry.

FIG. 8 shows lineage tracing revealing dhS1P-induced lymphocytes are not of myeloid-origin. Gr-1+CD11b+MDSC-like cells were isolated by high-speed cell sorting (85-95% purity) from the splenocytes of tumor-bearing MaFIA (Macrophage Fas-Induced Apoptosis) mice. These mice are on the C57BL/6J background and contain a transgene expressing both an inducible apoptosis feature as well as EGFP (enhanced green fluorescent protein). This transgene is expressed from the Csfr1 promoter (CD115), which restricts expression of thee transgene to the myeloid lineage. Isolated MDSC-like cells were exposed to dhS1P (5 μM) for 24 hours, and flow cytometry was performed to confirm both the disappearance of MDSC-like cells (Gr-1+CD11b+), and the appearance of a lymphocyte population (CD19+CD45R B220+). Lineage tracing using the EGFP feature of the transgene verified that dhS1P-induced lymphocytes (blue population) are EGFP negative and therefore not of myeloid-origin. This is in direct contrast with the EGFP positive MDSC-like cells (red population).

FIG. 9 (A-C) shows dihydrosphingosine-1-phosphate (dhS1P) exerts specific antitumor roles. (A) Splenic IMCs (Gr-1+CD11b+, also defined as MDSC: myeloid-derived suppressor cells) were isolated from subcutaneous human MDA-MB-231 breast tumor-bearing athymic nude mice, treated with or without dhS1P (to induce the expansion of CD19+CD45R B220+ cells: B-cells), and adoptively transferred into subcutaneous human MDA-MB-231 breast tumor-bearing athymic nude mice before monitoring tumor growth. ANOVA, **p<0.05 compared to PBS control, n≧6. (B) Splenic IMCs were isolated from orthotopic human BxPC-3 pancreatic tumor-bearing athymic nude mice, treated with or without dhS1P, and adoptively transferred into orthotopic human BxPC-3 pancreatic tumor-bearing athymic nude mice before monitoring survival. Logrank test, *p<0.05, n=5. (C) Tumor growth following BSA (lipid carrier control), sphingosine-1-phosphate (SIP), or dhS1P injection every other day, was monitored in C57BL/6J mice engrafted with subcutaneous murine Panc-02 pancreatic cancer cells. ANOVA, **p<0.05 compared to BSA control, n≧6.

### DETAILED DESCRIPTION OF THE PREFERRED EMBODIMENTS

The present invention now will be described more fully with reference to the accompanying examples. The invention may be embodied in many different forms and should not be construed as limited to the embodiments set forth in this application; rather, these embodiments are provided so that this disclosure will satisfy applicable legal requirements.

Many modifications and other embodiments of the invention will come to mind to one skilled in the art to which this invention pertains, having the benefit of the teachings presented in the descriptions and the drawings herein. As a result, it is to be understood that the invention is not to be limited to the specific embodiments disclosed and that modifications and other embodiments are intended to be included within the scope of the appended claims. Although specific terms are used in the specification, they are used in a generic and descriptive sense only and not for purposes of limitation.

The articles “a” and “an” are used to refer to one or more than one (i.e., to at least one) of the grammatical object of the article. By way of example, “an element” means one or more than one element.

### Myeloid Derived Suppressor Cells

The term “myeloid-derived suppressor cells” (“MDSC”s) as used herein refers to a heterogeneous population of immature myeloid cells expanded systemically as a consequence of a profound tumor-associated pro-inflammatory milieu, likely prematurely mobilized myeloid progenitors, and which have also been referred to as myeloid-derived suppressor cells. Immune suppressive cells are recognized in the art as critical cellular regulators by which tumors evade immunity and overcome therapeutic intervention. These suppressive cells include myeloid-derived suppressor cells (MDSC), which are immature myeloid cells with the ability to suppress immune effectors. In addition to tumors, MDSCs are also found at high numbers in the peripheral circulation and in organs such as the spleen and liver. MDSCs suppress T cell immunity via oxidative modification of the T cell receptor, and recent reports have shown that MDSCs can also impede dendritic (DC) and natural killer (NK) cell function. MDSCs increase as a function of tumor progression, and have been linked to the expansion of other immune suppressive cells such as regulatory T cells.

MDSC suppress immunity by perturbing both innate and adaptive immune responses. For example, MDSC block IL-2 production of anti-CD3-activated intratumoral T cells. These results have been confirmed in patients with a variety of cancers. MDSC also block the activation and proliferation of transgenic CD8+and CD4+ T cells cocultured with their cognate Ag. MDSC also suppress MHC allogeneic, Ag-activated CD4+ T cells, indicating that suppression may be nonspecific. Treatments that reduce MDSC levels such as antibody depletion of Gr1+ cells, treatment with the chemotherapeutic drug gemcitabine or retinoic acid, or the debulking of tumors restore immune surveillance, activate T and NK cells, and improve the efficacy of cancer vaccines or other immunotherapies in vivo. In vivo inactivation of genes that govern MDSC accumulation, such as the STAT3 and STAT6 genes and the nonclassical MHC class I CD1d gene, also restores T cell activation and promotes tumor regression and/or resistance to metastatic disease. Heightened cancer risk associated with aging is also attributed to the increasing levels of endogenous MDSC with advancing age, as is the increased growth rate of transplanted tumors in old vs. young mice. Collectively, these findings identify MDSC as a key cell population that prevents a host's immune system from responding to malignant cells. MDSC also indirectly effect T cell activation by inducing T regulatory cells (Tregs), which in turn down-regulate cell-mediated immunity. Treg induction may be induced by MDSC production of IL-10 and TGFβ, or arginase and is independent of TGFβ. MDSC can also suppress immunity by producing the type 2 cytokines, including for example IL-10, and/or by down-regulating macrophage production of the type 1 cytokine IL-12. This effect is amplified by macrophages that increase the MDSC production of IL-10. The role of MDSC in regulating NK cells is controversial. MDSC inhibit NK cell cytotoxicity against tumor cells and block NK production of IFN-γ, which requires cell contact between the MDSC and target cells. Suppression of NK cells may be mediated by blocking expression of NKG2D, a receptor on NK cells that is required for NK activation.

Tumor immunity may also be suppressed by interactions between MDSC and NKT cells. Type I (invariant or iNKT) NKT cells facilitate tumor rejection, whereas type II NKT cells promote tumor progression. Type II NKT cells facilitate tumor progression by producing IL-13, which induces the accumulation of MDSC and/or by polarizing macrophages toward a tumor-promoting M2-like phenotype.

In one aspect of the present invention, ICG-CPSNP PDT is employed as an anti-tumor effector, by inducing an immunomodulatory effect reducing MDSCs at the expense of increasing immune effectors. In a further aspect, ICG-CPSNP PDT is used to decrease the inflammatory milieu associated with MSDCs, for example by decreasing levels of IL-1β, IL-6, IL-12, IL-10, IFNγ, and/or TNFα. Examples of decreasing the inflammatory milieu further includes, for example, a decrease in the amount of an inflammatory mediator present, a decrease in the expression of an inflammatory mediator, a decrease in the activity of an inflammatory mediator, a decrease in response to inflammatory mediators or down regulation of receptors for inflammatory mediators, or a decrease in the activity of inflammation-associated transcription factors, for example NF-κB, HIF-1α, and STAT3 among others.

MDSCs typically bear the expression of multiple cell-surface markers that are normally specific for monocytes, macrophages or DCs and are comprised of a mixture of myeloid cells with granulocytic and monocytic morphology. Normal bone marrow contains 20-30% of IMCs, and IMCs make up small proportion (2-4%) of spleen cells. IMCs/MDSCs are not typically found in lymph nodes in mice. In humans, for healthy individuals, IMCs comprise ˜0.5% of peripheral blood mononuclear cells. In the case of cancer, IMCs specifically expanded and mobilized by tumor-associated factors exert an immunosuppressive phenotype that distinguishes them as MDSCs. Anticancer T-cell-dependent and -independent immune responses have been shown to be negatively regulated by MDSCs in a diversity of models of cancer. In addition to tumors, MDSCs are found at high numbers in the peripheral circulation and in organs such as the spleen and liver, and their systemic numbers are directly correlated with tumor burden. These immunosuppressive myeloid cells have been identified in both humans and mice, including athymic nude mice, with populations defined by the presence of particular combinations of surface antigens. In mice, MDSCs are Gr-1+CD11b+ granulocytic or monocytic cells, while in humans they are primarily defined within a CD14-HLA-DR-CD33+CD11b+ population. MDSCs can be identified by intrinsic features of NADPH oxidase activity, arginase activity, and/or nitric oxide synthase. Alternatively, MDSCs in mice can be identified by a Gr-1+ and/or CD11b+ phenotype. Because human cells do not express a marker homologous to mouse Gr1, they are typically phenotypically identified by a. In tumor tissues, MDSCs can be differentiated from tumor-associated macrophages (TAMs) by their high expression of Gr-1 (not expressed by TAMs) by their low expression of F4/80 (expressed by TAMs), by the fact that a large proportion of MDSCs have a granulocytic morphology and based the upregulated expression of both arginase and inducible nitric oxide synthase by MDSCs but not TAMs.

MDSC have been documented in most patients and mice with cancer, where they are induced by various factors produced by tumor cells and/or by host cells in the tumor microenvironment. In tumor-bearing mice MDSC accumulate in the bone marrow, spleen, and peripheral blood, within primary and metastatic solid tumors, and to a lesser extent in lymph nodes. In cancer patients MDSC are present in the blood, and potentially other sites. MDSC also accumulate in response to bacterial and parasitic infection, chemotherapy, experimentally induced autoimmunity, and stress. MDSC are considered a major contributor to the profound immune dysfunction of most patients with sizable tumor burdens. Cancers or individuals with cancer may be characterized as having high (or elevated) MDSC, low MDSC, or typical MDSC. This characterization may be based on quantification of cells in an individual or a tumor that bear the features or phenotypic identifiers of MDSC, for example by NADPH oxidase activity, arginase activity, and/or nitric oxide synthase, or Lin⁻FILA⁻DR⁻CD33⁺ and/or CD11b⁺CD14⁻CD33⁺ phenotype. This characterization may be made, for example, by assessing the percentage of tumor cells, splenocytes, or peripheral blood mononuclear cells that have MSDC identifiers. The characterization may also be made, for example, by determining the number of MDSC in a location, such as a tumor, spleen, or peripheral blood, and comparing to the number of MDSCs that would be observed in a similar location in a healthy individual. Alternatively, this characterization may be based on the inhibitory activity of the cells, including, for example, suppressing T cell immunity, impeding dendritic (DC) and natural killer (NK) cell function, and/or expansion of other immune suppressive cells such as regulatory T cells.

Immune suppression is an important aspect in the development and progression of cancer. Several suppressive immune cells have been described, with functional roles in a normal host that help to maintain a balanced immune response. Many studies have suggested that interaction between tumors and their microenvironments help to recruit immunosuppressive cells which can effectively block an antitumor response. Immune suppression can limit the efficacy of cancer therapy regimens. Intriguingly, MDSCs have been shown to regulate both T cell dependent and independent immune responses. Moreover, MDSCs have been described in a diversity of cancers and animal models of cancer, including tumor-bearing athymic nude mice. Specifically, MDSCs have been shown to be increased in laboratory models of cancer as well as cancer patients. These cells directly interfere with T cell mediated immunity, dendritic and natural killer cell function. Therefore significant effort is underway toward the development of therapies that decrease MDSCs. Surprisingly, the inventors have discovered that dhS1P can be useful in cancer therapy by decreasing a patient's MDSC count and/or stimulating the patient's immune system.

Without wishing to be bound by this theory, in one aspect of the invention, a previously described deep tissue imaging modality, which utilizes encapsulation of indocyanine green within a calcium phosphosilicate-matrix nanoparticle (ICG-CPSNP), can be utilized as an immunoregulatory therapeutic agent by increasing the amount of dhS1P available. The dhS1P can be exogenously supplied, for example delivered by CPSNPs, or can be increased endogenously.

The inventors have further discovered that the reduction of MDSCs by ICG-CPSNP Photodynamic Therapy (PDT) was dependent upon bioactive sphingolipids. Thus, in one aspect of the invention, ICG-CPSNP PDT, also referred to as PhotoImmunoNanoTherapy, may be used to induce a sphingosine kinase-dependent increase in dhS1P. PhotoImmunoNanoTherapy is described in United States Patent Pub. No. US 2010-0247436, titled In Vivo Photodynamic Therapy of Cancer via a Near Infrared Agent Encapsulated in Calcium Phosphate Nanoparticles, and is incorporated herein in its entirety. Specifically, Pub. No. US 2010-0247436 describes nano-encapsulated photosensitizers, wherein the photosensitizers are encapsulated in a calcium phosphate nanoparticle (CPNP), and their use in cancer treatment and/or imaging.

The inventors have found that isolated MDSCs are decreased by treatment with dhS1P, but not SIP, while dhS1P induces a concomitant expansion of antitumor B cells. In another aspect of the invention, these dhS1P-induced B cells can be adoptively transferred of into a patient, individual, or animal in need thereof to treat, block, or prevent cancer tumor growth. Collectively, these findings also reveal that PDT utilizing the combination therapeutic and diagnostic—or “theranostic”—agent ICG-CPSNP also behaved as a photo-immunotherapy in breast cancer by prompting a decrease in immunosuppressive MDSCs and an increase in immune effectors.

The inventors have developed novel therapies for cancer patients which decreases immunosuppressive MDSCs and permit the immune system to attack cancer cells. Using the methods described, one can utilize ICG-CPSNP PDT to directly treat the tumor area and decrease the immunosuppression caused by the cancer cells, one can also directly treat patients with dhS1P and decrease the immunosuppression, and one can also expose MDSCs to dhS1P and then transfer the resultant dhS1P-induced B cells to a patient in need of cancer therapy.

### Sphingolipids

Sphingolipids are an extensive classification of lipids which play prominent roles in cellular signaling in addition to being essential components of membranes. As used herein, “sphingolipids” refers to lipids containing a backbone of sphingoid bases. Examples of sphingolipids include sphingosine, dihydrosphingosine, sphingosine-1-phosphate (S1P), dihydrosphingosine-1-phosphate (dhS1P), phytosphingosine, ceramide, dihydroceramide, ceramide-1-phosphate, phytoceramide, sphingomyelin, glycosphingolipids, cerebrosides, sulfatides, gangliosides, and inositol-containing ceramides. Sphingolipids play profound roles in cellular survival, mitogenesis, proliferation, death, and signaling. Different sphingolipids are noteworthy for regulating specific biological effects. The most well studied sphingolipid is ceramide, an N-acylated sphingosine, which serves as a hypothetical center of sphingolipid metabolism. Much attention has been given to the role of ceramides in the induction of cell death, and in particular in response to chemotherapy, radiation therapy, and even PDT. More so, recently designed nanoliposomes containing ceramide analogs have proven efficacious in treating several models of cancer. Many chemotherapeutics, radiation therapy, and PDT, have been shown to increase levels of the sphingolipid ceramide in cancerous tissue, while relapsing and therapy resistant cancers possess the inherent ability to detoxify ceramide to neutral or pro-oncogenic phosphorylated metabolites.

On the other side of the death versus survival spectrum from ceramide lies the metabolically related sphingolipid S1P. The roles of S1P have been primarily ascribed to survival, proliferation, and mitogenesis, but also to regulation of the immune system. In particular, S1P has been shown to regulate the trafficking of immune effectors. The conversion of ceramide to sphingosine-1-phosphate (S1P) has been extensively studied namely due to ceramide's role as a pro-apoptotic, pro-cellular stress, anti-inflammatory lipid and S1P's role as a pro-survival, mitogenic, and oncogenic lipid. S1P has also been shown to be immunogenic, stimulating cells of the immune system and promoting their trafficking, via binding to S1P G protein-coupled receptors. In cancer, sphingolipids such as S1P are often elevated, while ceramides are decreased, providing an environment friendly to tumor growth.

According to an aspect of the invention, the sphingolipid of the present invention is dhSpl or an analog or derivative thereof. The dhS1P or analog or derivative thereof according to the present invention encompasses any lipid containing a backbone of sphingoid bases that exhibits an anticancer effect, including by decreasing the number of MDSCs and/or increasing the number of B-cells. According to a further aspect of the invention, the sphingolipids can be a sphingolipid with one of the following formulas:

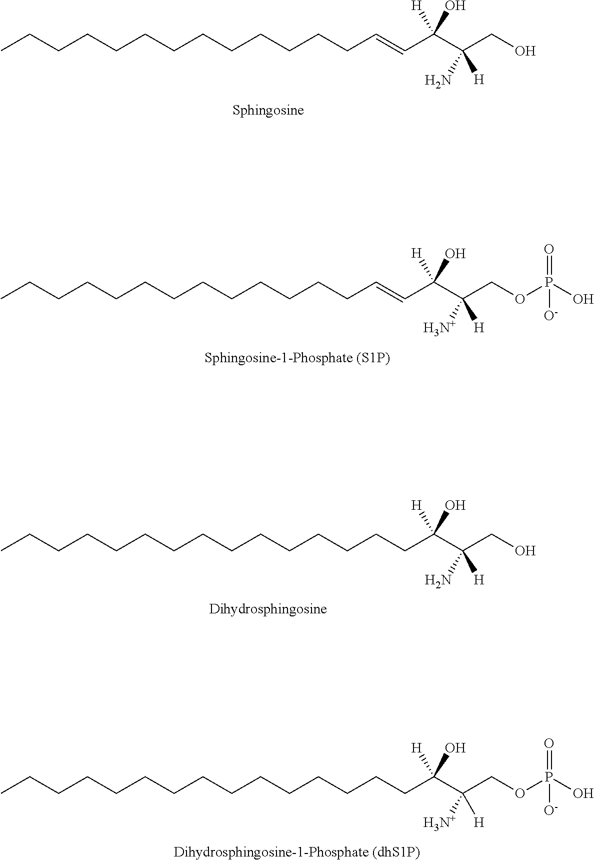 [SMILES: CCCCCCCCCCCCC/C=C/[C@@H](O)[C@@H](N)CO ; CCCCCCCCCCCCC/C=C/[C@@H](O)[C@@H]([NH3+])COP(=O)([O-])O ; CCCCCCCCCCCCCCC[C@@H](O)[C@@H](N)CO ; CCCCCCCCCCCCCCC[C@@H](O)[C@@H]([NH3+])COP(=O)([O-])O]

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. As defined herein, the term “derivative” refers to compounds that have a common core structure, and are substituted with various groups as described herein.

In one aspect of the invention, PhotoImmunoNanoTherapy, including ICG-CPSNP PDT, alters phosphorylated sphingolipid metabolites. In a further aspect PhotoImmunoNanoTherapy induces a specific increase in SIP and dhS1P. This increase induces antitumor activity. In a further aspect, PhotoImmunoNanoTherapy induces an increased in mass levels of phosphorylated sphingolipid, for example through a release of phosphorylated sphingolipids from tumor or cancer cells in response to PhotoImmunoNanoTherapy.

In another aspect of the invention, dhS1P, or analogs or derivatives thereof, can be administered directly, thereby exerting an anticancer effect, including by decreasing the number of MDSCs and increasing the number of B-cells in a subject with cancer.

### Cancer and Tumor Types

Compositions and methods of the present invention may be used to treat any number of cancers. According to an embodiment of the invention dhS1P, which is responsible for the antitumor effect of ICG-CPSNP PDT, is used in compositions and methods for treating a wide variety of cancer types. The terms “cancer” and “tumor” are used interchangeably, and as used herein refer to the commonly understood spectrum of diseases including, but not limited to, solid tumors, such as cancers of the breast, respiratory tract, brain, reproductive organs, digestive tract, urinary tract, eye, liver, skin, head and neck, thyroid, parathyroid and their distant metastases, and also includes lymphomas, sarcomas, and leukemias. Examples of breast cancer include, but are not limited to invasive ductal carcinoma, invasive lobular carcinoma, ductal carcinoma in situ, and lobular carcinoma in situ. Examples of cancers of the respiratory tract include, but are not limited to small-cell and non-small-cell lung carcinoma, as well as bronchial adenoma and pleuropulmonary blastoma. Examples of brain cancers include, but are not limited to brain stem and hypophthalmic glioma, cerebellar and cerebral astrocytoma, medulloblastoma, ependymoma, as well as neuroectodermal and pineal tumor. Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus. Tumors of the digestive tract include, but are not limited to anal, colon, colorectal, esophageal, gallbladder, gastric, pancreatic, rectal, small intestine, and salivary gland cancers. Tumors of the urinary tract include, but are not limited to bladder, penile, kidney, renal pelvis, ureter, and urethral cancers. Eye cancers include, but are not limited to intraocular melanoma and retinoblastoma. Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma. Skin cancers include, but are not limited to squamous cell carcinoma, Kaposi's sarcoma, malignant melanoma, Merkel cell skin cancer, and non-melanoma skin cancer. Head-and-neck cancers include, but are not limited to laryngeal/hypopharyngeal/nasopharyngeal/oropharyngeal cancer, and lip and oral cavity cancer. Lymphomas include, but are not limited to AIDS-related lymphoma, non-Hodgkin's lymphoma, cutaneous T-cell lymphoma, Hodgkin's disease, and lymphoma of the central nervous system. Sarcomas include, but are not limited to sarcoma of the soft tissue, fibrosarcoma, osteosarcoma, malignant fibrous histiocytoma, lymphosarcoma, and rhabdomyosarcoma. Leukemias include, but are not limited to acute myeloid leukemia, acute lymphoblastic leukemia, chronic lymphocytic leukemia, chronic myelogenous leukemia, and hairy cell leukemia. Cancers also specifically include, but are not limited to, chronic myeloid leukemia (CML), acute myeloid leukemia (AML), cutaneous T cell lymphoma (CTCL), cutaneous T cell lymphoma (CTCL), acute T lymphoblast leukemia (ALL), MDR acute T lymphoblast leukemia (MDR ALL), large B-lymphocyte non-Hodgkin's lymphoma, leukemic monocyte lymphoma, epidermal squamous carcinoma, epithelial lung adenocarcinoma, liver hepatocellular carcinoma, colorectal carcinoma, breast adenocarcinoma, brain glioblastoma, prostate adenocarcinoma, gastric carcinoma and other cancerous tissues. Cancers further include all forms of cancer expressing lysine specific demethylase 1 (LSD1). These disorders have been characterized in humans, but also exist with a similar etiology in other mammals, and can be treated by administering the methods and compositions of the present invention.

In one aspect of the invention, a robust antitumor immune response is induced, for example through dhS1P-dependent reduction in MDSC-like cells and/or a concomitant increase in immune effectors. In an aspect of the invention the antitumor response is induced by administration of dhS1P. In another aspect of the invention the antitumor response is induced by PhotoImmunoNanoTherapy. In another embodiment of the invention the antitumor effect is induced by ICG-CPSNP PDT in low oxygen tumor environments.

It is understood that the ability of dhS1P to reduce MDSC cells, provides a basis from which to predict efficacy for all types of tumors or cancer where elevated levels of MDSCs or IMCs are observed. Elevated MDSC levels include tumor types where the number of MDSCs (as measured by any technique known in the art) is higher than the number of MDSCs that would be observed in a similar location in a healthy individual. Elevated MDSCs are present in most cancer patients, including, for example, patients with squamous cell carcinomas; breast, head and neck, and lung cancer; metastatic adenocarcinomas of the pancreas, colon, and breast; renal-cell carcinomas; prostate cancer; nonsmall cell lung cancer; multiple myeloma; brain tumors and gliomas; melanoma; leukemia; lymphomas; eye tumors; gastrointestinal cancer; thyroid cancer, including anaplastic thyroid carcinoma; hepatocellular carcinoma; malignant melanoma; chronic myeloid leukemia; and acute myeloid leukemia.

### Inflammation in Cancer and Cancer Treatment

Inflammation is characteristic of cancer and the tumor microenvironment, and represents a crucial player in the tumor development and progression. Both extrinsic and intrinsic pathways of cancer-related inflammation activate transcription factors (mainly NF-κB, HIF-1α, STAT3) which are the key inducers of inflammatory mediators (e.g. cytokines chemokines, prostaglandins and nitric oxide (NO)). Examples of inflammatory mediators that are part of the inflammatory milieu of cancer and/or tumors include the pro-inflammatory S-1 00 protein, CSF-1, IL-6, IL-10, VEGF, IL-1β, IL-6, IL-12, IL-10, IFNγ, and/or TNFα. According to one aspect of the invention, compositions of the invention are used to decrease the inflammatory milieu associated with MSDCs, for example by decreasing levels of IL-1β, IL-6, IL-12, IL-10, IFNγ, and/or TNFα. For example, a decrease in the inflammatory milieu associated with MSDCs can be obtained through delivery dhS1P or through delivery of ICG-CPSNP and PDT.

### Compositions

Compositions containing dhS1P may be formulated in any conventional manner. Proper formulation is dependent upon the route of administration chosen. Suitable routes of administration include, but are not limited to, oral, parenteral (e.g., intravenous, intra-arterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Pharmaceutically acceptable carriers for use in the compositions of the present invention are well known to those of ordinary skill in the art and are selected based upon a number of factors: dhS1P concentration and intended bioavailability; the disease, disorder or condition being treated with the composition; the subject, his or her age, size and general condition; and the route of administration. Suitable carriers are readily determined by one of ordinary skill in the art (see, for example, J. G. Nairn, in: Remington's

Pharmaceutical Science (A. Gennaro, ed.), Mack Publishing Co., Easton, Pa., (1985), pp. 1492-1517, the contents of which are incorporated herein by reference). For oral administration, the compositions containing dhS1P are preferably formulated as tablets, dispersible powders, pills, capsules, gelcaps, caplets, gels, liposomes, granules, solutions, suspensions, emulsions, syrups, elixirs, troches, dragees, lozenges, or any other dosage form which can be administered orally. Techniques and compositions for making oral dosage forms useful in the present invention are described in the following references: 7 Modern Pharmaceutics, Chapters 9 and 10 (Banker & Rhodes, Editors, 1979); Lieberman et al., Pharmaceutical Dosage Forms: Tablets (1981); and Ansel, Introduction to Pharmaceutical Dosage Forms 2nd Edition (1976).

Suitable carriers used in formulating liquid dosage forms for oral or parenteral administration include nonaqueous, pharmaceutically-acceptable polar solvents such as oils, alcohols, amides, esters, ethers, ketones, hydrocarbons and mixtures thereof, as well as water, saline solutions, dextrose solutions (e.g., DW5), electrolyte solutions, or any other aqueous, pharmaceutically acceptable liquid.

Suitable nonaqueous, pharmaceutically-acceptable polar solvents include, but are not limited to, alcohols (e.g., .alpha.-glycerol formal, .beta.-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(.beta.-hydroxyethyl)-lactamide, N,N-dimethylacetamide amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)30-60 sorbitol poly(oleate)2-4, poly(oxyethylene)15-20 monooleate, poly(oxyethylene)15-20 mono 12-hydroxystearate, and poly(oxyethylene)15-20 mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C4-C22 fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

In addition to human subjects, the present invention may be applied to non-human animals, such as mammals, particularly those important to agricultural applications (such as, but not limited to, cattle, sheep, horses, and other “farm animals”), industrial applications (such as, but not limited to, animals used to generate bioactive molecules as part of the biotechnology and pharmaceutical industries), and for human companionship (such as, but not limited to, dogs and cats).

### Use of PhotoImmunoNanoTherapy

U.S. Patent Pub. No. US 2010-0247436, which is incorporated herein in its entirety, discloses successful targeting of ICG-loaded CPSNPs to leukemia stem cells allowed for successful in vivo PDT of chronic myeloid leukemia. In one embodiment of the invention, these treatment modalities can be stand-alone treatments or as part of adjuvant, neoadjuvant and/or concomitant therapy with one or more other cancer treatments. In one aspect, PDT utilizing ICG-CPSNPs can be employed as a “theranostic” modality for solid tumors) and that its efficacy is due, at least in part, to regulation of the immune milieu.

### Methods of Administration

Direct Administration of dhS1P

Compositions of the present invention include dhS1P, or analogs or derivatives thereof. For topical administration, the dhS1P may be in standard topical formulations and compositions including lotions, suspensions or pastes. dhS1P may be administered by various means, but preferably by intravenous injection.

The experimental data disclosed in this application, in direct contradiction to the commonly held assumptions regarding dhS1P, demonstrate that dhS1P results in a decrease in MDSCs and is effective in the treatment of cancer. The decrease in MDSCs results in an increase in immune activity characterized by an expansion of B cells which is unexpected considering that the related lipid S1P is oncogenic and that its immunomodulatory aspects are mainly limited to the trafficking of a wide diversity of immune cells and progenitors. For these and other reasons there is a need for the present invention.

Without wishing to be bound by any particular theory, the inventors have found that dhS1P exerts an anticancer effect, including by decreasing the number of MDSCs and increasing the number of B-cells in a subject with cancer. In particular, the inventors have demonstrated that dhS1P causes the ablation of MSDCs. A person of skill in the art would understand that these effects can be achieved through administration of dhS1P. In one aspect, dhS1P, or analogues or derivatives thereof, can be administered directly to an individual, subject, patient, or animal, either systemically or to the site of the cancer or tumor. In another aspect, dhS1P or analogues or derivatives thereof, can be delivered encapsulated in CPSNPs, either systemically or to the site of the cancer or tumor. In another aspect, dhS1P can be increased endogenously in the individual, subject, patient, or animal, for example through induction by ICG-CPSNP PDT.

In another aspect, the methods include administering systemically or locally the photosensitizer-encapsulated nanoparticles of the invention. The photosensitizer-encapsulated nanoparticle may further comprise dhS1P, or may be given in conjunction with dhS1P. Methods for preparing nanoparticles and encapsulating compounds are disclosed in Pub. No. US 2010-0247436. It is understood that these methods can be used for the encapsulation and delivery of dhS1P. In another aspect of the invention, the photosensitizer-encapsulated nanoparticles of the invention, for example ICG-CPSNPs, are used to induce an increase of endogenous dhS1P through PDT.

Any suitable route of administration may be used for delivery of dhS1P, either directly or encapsulated in CPSNPs, including, for example, topical, intravenous, oral, subcutaneous, local (e.g. in the eye) or by use of an implant. Advantageously, the small size, colloidal stability, non-agglomeration properties, and enhanced half-life of the nanoparticles render the nano-encapsulated photosensitizer especially suitable for intravenous administration. Additional routes of administration are subcutaneous, intramuscular, or intraperitoneal injections in conventional or convenient forms.

The dose of dhS1P may be optimized by the skilled person depending on factors such as, but not limited to, the nature of the therapeutic protocol, the individual subject, and the judgment of the skilled practitioner. Preferred amounts of dhS1P are those which are clinically or therapeutically effective in the treatment method being used. Such amounts are referred herein as “effective amounts”.

Depending on the needs of the subject and the constraints of the treatment method being used, smaller or larger doses of dhS1P may be needed. The doses may be a single administration or include multiple dosings over time. The preferred dosage range for use in humans or mice is from 0.001 mg/kg to 1 mg/kg, however the preferred minimum therapeutic amount in the dosage range can be 0.001, 0.01, 0.02, 0.03, 0.04, 0.05, 0.06, 0.07, 0.08, 0.09, 0.1, 0.2, 0.3, 0.4, 0.5, 0.6, 0.7, 0.8 or 0.9 mg/kg, likewise, the maximum preferred therapeutic amount in the dosage range can be 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9 or 1.0 mg/kg. Serum levels measured in the experiments were generally around 0.005 mg/kg. The foregoing ranges are merely suggestive in that the number of variables with regard to an individual treatment regime is large and considerable deviation from these values may be expected. The skilled artisan is free to vary the foregoing concentrations so that the uptake and stimulation/restoration parameters are consistent with the therapeutic objectives disclosed above. Administration and dosing of photosensitizer-encapsulated nanoparticles, including for example ICG-CPSNPs, is disclosed in Pub. No. US 2010-0247436.

### Methods of Treatment

Treatment with PhotoImmunoNanoTherapy

Methods of treatment using PhotoImmunoNanoTherapy are described in U.S. Patent Pub. No. 2010-0247436. According to an aspect of the present invention, these methods can be used to decrease the number of MSDCs. In one embodiment, PhotoImmunoNanoTherapy may be used to induce an increase in dhS1P. In a preferred embodiment, PhotoImmunoNanoTherapy may be used to treat cells in culture to induce an increase in dhS1P, thereby decreasing the number of MSDCs and/or increasing the number of B cells, which may then be administered to an individual, subject, or patient in need thereof. In another preferred embodiment, PhotoImmunoNanoTherapy may be used to treat an individual, patient, or subject by administering nanoparticles, for example ICG-CPSNP, to a tumor, specific location, or systemically, and subsequent PDT, thereby inducing an increase in dhS1P and a decrease of MDSC in the individual, subject, or patient. The route of administration of the nanoparticles may be topically, intravenously, orally, locally, subcutaneously, intramuscularly, or intraperitoneally.

Treatment with dhS1P

In another aspect of the invention, treatment may be accomplished by direct administration of dhS1P. According to one embodiment, dhS1P may be used to treat cells in culture to decrease the number of MSDCs and/or increase the number of B cells, which may then be administered to an individual, subject, or patient in need thereof. In another embodiment, dhS1P may be used to treat an individual, subject, or patient, for example, by administering dhS1P to a tumor, specific location, or systemically, thereby inducing a decrease of MDSC in the individual, subject, or patient. The route of administration may be topically, intravenously, orally, locally, subcutaneously, intramuscularly, or intraperitoneally.

### Cancer Therapy Agents

The compositions and methods according to the invention may also employ a cancer therapy or chemotherapeutic agent. As used herein, the terms “cancer therapy,” “cancer therapeutic,” “chemotherapy” and “chemotherapeutic” are used interchangeably, and refer to agents that are customarily employed to diminish cell proliferation and/or to induce cell apoptosis as one skilled in the art appreciates. Additional cancer therapies may also be employed in combination with ICG-CPSNPS and dhS1P according to the invention, including for example biotherapeutic agents, radiopharmaceuticals, and the like.

According to the invention, the term “cancer therapy,” “cancer therapeutic,” “chemotherapy” and “chemotherapeutic” includes both the killing of tumor cells, the reduction of the proliferation of tumor cells (e.g. by at least 30%, at least 50% or at least 90%) as well as the complete inhibition of the proliferation of tumor cells. Furthermore, this term includes the prevention of a tumorigenic disease, e.g. by killing of cells that may or are prone to become a tumor cell in the future as well as the formation of metastases.

According to the invention, administration of dhS1P may be in combination with another cancer therapy. This combination may include any combined administration of the dhS1P and the cancer therapy. This may include the simultaneous application of dhS1P and the cancer therapy or, preferably, a separate administration. The term “concomitant therapy” refers to the simultaneous application of dhS1P and the cancer therapy, or application in rapid succession. In case that a separate administration is envisaged, one would preferably ensure that a significant period of time would not expire between the times of delivery, such that dhS1P and the cancer therapy would still be able to exert an advantageously combined effect on cancer. In such instances, it is preferred that one would administer both agents within about one week, preferably within about 4 days, more preferably within about 12-36 hours of each other. The rationale behind this aspect of the invention is that administration of dhS1P prevents the immunosuppressive activity of MSDC makes the tumor cells a better target for the cancer therapy, in particular cancer immunotherapy. Therefore, this aspect of the invention also encompasses treatment regimens where dhS1P is administered in combination with the cancer therapy in various treatment cycles wherein each cycle may be separated by a period of time without treatment which may last, for example, for two weeks and wherein each cycle may involve the repeated administration of dhS1P and/or the cancer therapy. For example such treatment cycle may encompass the treatment with dhS1P, followed by a cancer therapy, for example a cancer immunotherapy within 2 days. Especially in the course of such repeated treatment cycles, it is also envisaged within the present invention that the dhS1P prior to the cancer therapy.

Throughout the invention, the skilled person will understand that the individual therapy to be applied will depend on the e.g. physical conditions of the patient or on the severity of the disease and will therefore have to be adjusted on a case to case basis.

As one skilled in the art appreciates, cancer chemotherapeutic agents are used for their lethal action to cancer cells. Unfortunately, few such drugs differentiate between a cancer cell and other proliferating cells. Chemotherapy generally requires use of several agents concurrently or in planned sequence. Combining more than one agent in a chemotherapeutic treatment protocol allows for: (1) the largest possible dose of drugs; (2) drugs that work by different mechanisms; (3) drugs having different toxicities; and (4) the reduced development of resistance. Chemotherapeutic agents mainly affect cells that are undergoing division or DNA synthesis, thus slow growing malignant cells, such as lung cancer or colorectal cancer, that are often unresponsive. Furthermore, most chemotherapeutic agents have a narrow therapeutic index. Common adverse effects of chemotherapy include vomiting, stomatitis, and alopecia. Toxicity of the chemotherapeutic agents is often the result of their effect on rapidly proliferating cells, which are vulnerable to the toxic effects of the agents, such as bone marrow or from cells harbored from detection (immunosuppression), gastrointestinal tract (mucosal ulceration), skin and hair (dermatitis and alopecia).

Many potent cytotoxic agents act at specific phases of the cell cycle (cell cycle dependent) and have activity only against cells in the process of division, thus acting specifically on processes such as DNA synthesis, transcription, or mitotic spindle function. Other agents are cell cycle independent. Susceptibility to cytotoxic treatment, therefore, may vary at different stages of the cell life cycle, with only those cells in a specific phase of the cell cycle being killed. Because of this cell cycle specificity, treatment with cytotoxic agents needs to be prolonged or repeated in order to allow cells to enter the sensitive phase. Non-cell-cycle-specific agents may act at any stage of the cell cycle; however, the cytotoxic effects are still dependent on cell proliferation. Cytotoxic agents thus kill a fixed fraction of tumor cells, the fraction being proportionate to the dose of the drug treatment.

Exemplary chemotherapeutic agents suitable for use in compositions and/or combinational therapies according to the invention include: anthracyclines, such as doxorubicin, alkylating agents, nitrosoureas, antimetabolites, such as 5-FU, platins, antitumor antibiotics, such as dactinomycin, daunorubicin, doxorubicin (Adriamycin), idarubicin, and mitoxantrone, miotic inhibitors, alkylating agents, mitotic inhibitors, steroids and natural hormones, including for example, corticosteroid hormones, sex hormones, immunotherapy or others such as L-asparaginase and tretinoin. These and other specific examples of chemotherapeutic agents are well known to those of skill in the art and are included within the scope of the invention.

### Cancer Immunotherapy

Cancer immunotherapy is therapy which is intended to stimulate a patient's immune system to attack the tumor cells. Cancer immunotherapy can be accomplished through the use a number of means including the use of immunization technologies (such as cancer vaccines) and the administration of therapeutic antibodies. Depending on the approach used, the patient's immune system is either trained to recognize tumor cells as targets for destruction (e.g. immunization therapies) or recruited to destroy tumor cells (e.g. therapeutic antibodies). Immunotherapy can help the immune system recognize cancer cells, or enhance a response against cancer cells. Immunotherapies include active and passive immunotherapies. Active immunotherapies stimulate the body's own immune system while passive immunotherapies generally use immune system components created outside of the body.

The premise behind cancer immunotherapy is that many tumor cells display unusual antigens which are either inappropriate for the particular cell type or are not normally present at the patients current level of development (e.g. fetal antigens). The effectiveness of such immunotherapies can be limited by immunosuppressive tumor environments. Thus improved techniques of modulating the immunosuppressive environment of tumors are required. The inventors have discovered that dhS1P decreases the MDSC population, reducing the immunosuppressive environment. By modulating the immune suppression, administration of dhS1P clears the way for increased effectiveness of cancer immunotherapy approaches.

In one embodiment, the compounds of the invention can be used in combination with an immunotherapeutic agent for the treatment of a proliferative disorder such as cancer, or to prevent the reoccurrence of a proliferative disorder such as cancer. The term “immunotherapy agent,” “immunotherapeutic,” “immunotherapeutic agent,” and “immunotherapy” are used interchangeably (also called biological response modifier therapy, biologic therapy, biotherapy, immune therapy, or biological therapy) and refer to treatment that uses parts of the immune system to fight disease. Examples of active immunotherapy agents include: cancer vaccines, tumor cell vaccines (autologous or allogeneic), viral vaccines, dendritic cell vaccines, antigen vaccines, anti-idiotype vaccines, DNA vaccines, Lymphokine-Activated Killer (LAK) Cell Therapy, or Tumor-Infiltrating Lymphocyte (TIL) Vaccine with Interleukin-2 (IL-2). Active immunotherapy agents are currently being used to treat or being tested to treat various types of cancers, including melanoma, kidney (renal) cancer, bladder cancer, prostate cancer, ovarian cancer, breast cancer, colorectal cancer, lung cancer, leukemia, prostate cancer, non-Hodgkin's lymphoma, pancreatic cancer, lymphoma, multiple myeloma, head and neck cancer, liver cancer, malignant brain tumors, and advanced melanoma.

Examples of passive immunotherapy agents include: monoclonal antibodies and targeted therapies containing toxins. Monoclonal antibodies include naked antibodies and conjugated antibodies (also called tagged, labeled, or loaded antibodies). Naked monoclonal antibodies do not have a drug or radioactive material attached whereas conjugated monoclonal antibodies are joined to a chemotherapy drug (chemolabeled), a radioactive particle (radiolabeled), or a toxin (immunotoxin). A number of naked monoclonal antibody drugs have been approved for treating cancer, including:

Rituximab (Rituxan), an antibody against the CD20 antigen used to treat B cell non-Hodgkin lymphoma; Trastuzumab (Herceptin), an antibody against the HER2 protein used to treat advanced breast cancer; Alemtuzumab (Campath), an antibody against the CD52 antigen used to treat B cell chronic lymphocytic leukemia (B-CLL); Cetuximab (Erbitux), an antibody against the EGFR protein used in combination with irinotecan to treat advanced colorectal cancer and to treat head and neck cancers; and Bevacizumab (Avastin) which is an antiangiogenesis therapy that works against the VEGF protein and is used in combination with chemotherapy to treat metastatic colorectal cancer. A number of conjugated monoclonal antibodies have been approved for treating cancer, including: Radiolabeled antibody Ibritumomab tiuxetan (Zevalin) which delivers radioactivity directly to cancerous B lymphocytes and is used to treat B cell non-Hodgkin lymphoma; radiolabeled antibody Tositumomab (Bexxar) which is used to treat certain types of non-Hodgkin lymphoma; and immunotoxin Gemtuzumab ozogamicin (Mylotarg) which contains calicheamicin and is used to treat acute myelogenous leukemia (AML). BL22 is a conjugated monoclonal antibody currently in testing for treating hairy cell leukemia and there are several immunotoxin clinical trials in progress for treating leukemias, lymphomas, and brain tumors. There are also approved radiolabeled antibodies used to detect cancer, including OncoScint for detecting colorectal and ovarian cancers and ProstaScint for detecting prostate cancers. Targeted therapies containing toxins are toxins linked to growth factors and do not contain antibodies. An example of an approved targeted therapy containing toxins is denileukin diftitox (Ontak) which is used to treat a type of skin lymphoma (cutaneous T cell lymphoma).

Examples of adjuvant immunotherapies include: cytokines, such as granulocyte-macrophage colony-stimulating factor (GM-CSF), granulocyte-colony stimulating factor (G-CSF), macrophage inflammatory protein (MIP)-1-alpha, interleukins (including IL-1, IL-2, IL-4, IL-6, IL-7, IL-12, IL-15, IL-18, IL-21, and IL-27), tumor necrosis factors (including TNF-alpha), and interferons (including IFN-alpha, IFN-beta, and IFN-gamma); aluminum hydroxide (alum); Bacille Calmette-Guerin (BCG); Keyhole limpet hemocyanin (KLH); Incomplete Freund's adjuvant (IFA); QS-21; DETOX; Levamisole; and Dinitrophenyl (DNP). Clinical studies have shown that combining IL-2 with other cytokines, such as IFN-alpha, can lead to a synergistic response.

The term “neoadjuvant” refers to the administration of therapeutic agents before a main treatment. Neoadjuvant therapy aims to reduce the size or extent of the cancer before using radical treatment intervention, thus making procedures easier and more likely to succeed, and reducing the consequences of a more extensive treatment technique that would be required if the tumor wasn't reduced in size or extent. The use of therapy can turn a tumour from untreatable to treatable by shrinking the volume down.

The development and utilization of ICG-CPSNPs initially was postulated to improve diagnostic imaging for breast cancer. Intriguingly, this advancement in imaging with ICG-CPSNPs also overcame limitations associated with traditional PDT. Based upon the improved quantum efficiency and improved half-life, it was hypothesized that ICG-CPSNPs could be used as a combination therapeutic and diagnostic—or “theranostic”—modality for cancer. According to one aspect of the invention PhotoImmunoNanoTherapy may be employed to prevent or block development of cancer and/or prevent or block tumor growth. In one embodiment, the therapy comprises administration ICG-CPSNP. Administration may be performed as described above. Further, PhotoImmunoNanoTherapy according to an embodiment of the invention may be employed for long-term blockage of cancer or tumor development. Further still, PhotoImmunoNanoTherapy according to an embodiment of the invention may be employed to promote an anti-cancer immune response. Further still, PhotoImmunoNanoTherapy according to an embodiment of the invention may be employed in conjunction with additional cancer therapy, including, for example, cancer immunotherapy.

The inventions being thus described, it will be obvious that the same may be varied in many ways. Such variations are not to be regarded as a departure from the spirit and scope of the inventions.

### EXAMPLES

### Example 1

### PhotoImmunoNanoTherapy Blocks Tumor Progression and Extends Survival

The efficacy of dhS1P and PhotoImmunoNanoTherapy was evaluated in two murine models of breast cancer to study effects in T-cell-competent hosts (murine 410.4 cells in BALB/cJ mice), and T-cell-deficient hosts (human MDA-MB-231 cells in athymic nude mice; murine 410.4 cells in NOD.CB 17-Prkdc^{scid}/J mice), in addition to a subcutaneously engrafted model of pancreatic cancer (murine Panc-02 cells in immunocompetent C57BL/6J mice), an orthotopic pancreatic cancer model (human BxPC-3 cells in athymic nude mice), and an experimental model of lung-metastatic osteosarcoma (human SAOS-2-LM7 cells in athymic nude mice). A robust antitumor immune response was observed, and demonstrated to be due to dhS1P-dependent reduction in MDSC-like cells and a concomitant increase in immune effectors. Thus, immunomodulation was implicated as a critical mechanism by which ICG-CPSNP PDT can exert an antitumor effect in low oxygen tumor environments.

To evaluate the antitumor efficacy of PhotoImmunoNanoTherapy, two murine models of breast cancer were utilized to study effects in T-cell-competent hosts (murine 410.4 cells in BALB/cJ mice) and T-cell-deficient hosts (human MDA-MB-231 cells in athymic nude mice; murine 410.4 cells in NOD.CB17-Prkdcscid/J mice), in addition to a subcutaneously engrafted model of pancreatic cancer (murine Panc-02 cells in C57BL/6J mice), an orthotopic pancreatic cancer model (human BxPC-3 cells in athymic nude mice), and an experimental model of lung-metastatic osteosarcoma (human SAOS-2-LM7 cells in athymic nude mice). Treatments were initiated one week following tumor establishment and consisted of injections of ICG-CPSNPs or controls followed 24 h later by NIR laser treatment of the tumor location to allow adequate tumor accumulation of PEGylated ICGCPSNPs. Tumor growth was effectively blocked and survival extended by

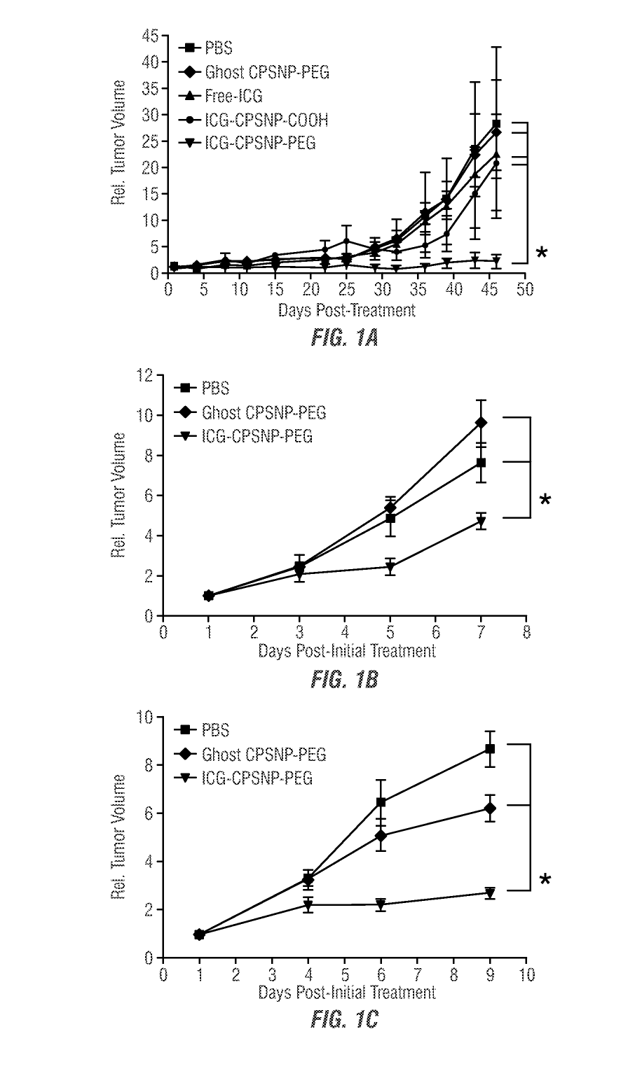

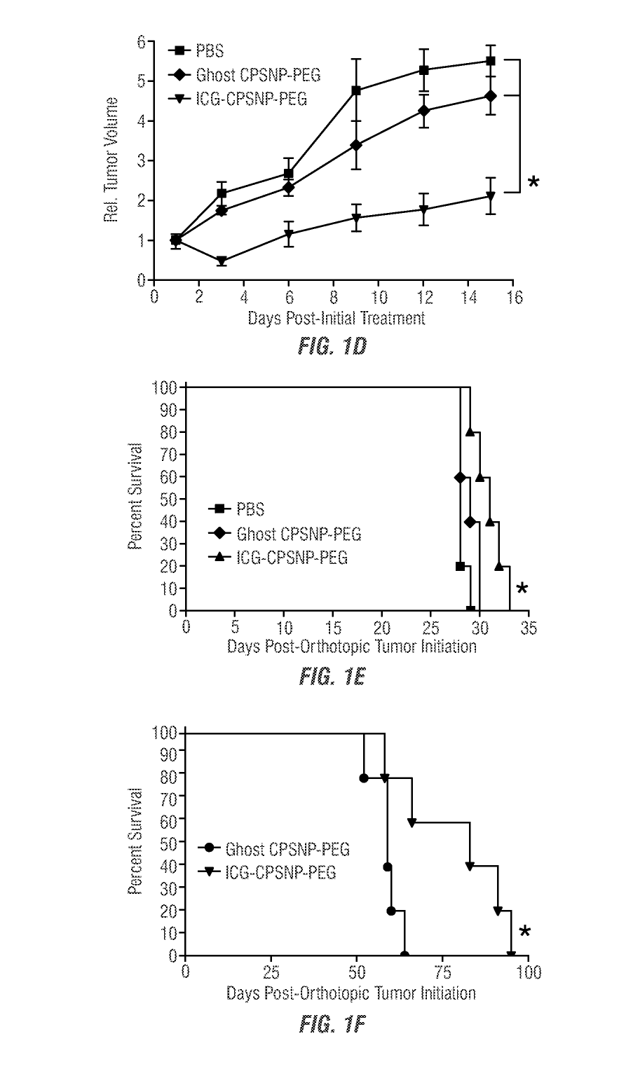

PhotoImmunoNanoTherapy in (FIG. 1A-F): (1) human MDA-MB-231 cells in athymic nude mice (subcutaneous), (2) murine 410.4 breast cancer cells in BALB/cJ mice (subcutaneous), (3) murine 410.4 breast cancer cells in NOD.CB17-Prkdcscid/J mice (subcutaneous), (4) murine Panc-02 pancreatic cancer cells in C57BL/6J mice (subcutaneous), (5) human BxPC-3-GFP pancreatic cancer cells in athymic nude mice (orthotopic), and (6) human SAOS-2-LM7 osteosarcoma cells in athymic nude mice (experimental lung metastases). In the most elaborate study, MDA-MB-231 tumor growth was abrogated in athymic nude mice receiving PEGylated ICG-CPSNPs but not PBS or PEGylated ghost CPSNPs (FIG. 1A). Furthermore, MDA-MB-231 tumor growth was not blocked by non-PEGylated (citrate-terminated) ICG-CPSNPs or free ICG. This observation is consistent with previous findings which demonstrated that only PEGylated ICG-CPSNPs, but not non-PEGylated ICG-CPSNPs or free ICG, accumulated within MDA-MB-231 tumors, indicating that the presence of ICG-CPSNPs within tumors is required for antitumor efficacy of PhotoImmunoNanoTherapy. Long-term blockade of tumor growth with a minimal treatment suggested a possible antitumor immune response, while the efficacy in athymic nude mice and NOD.CB17-Prkdcscid/

### Example 2

### MDSCs are Decreased by ICG-CPSNP PDT

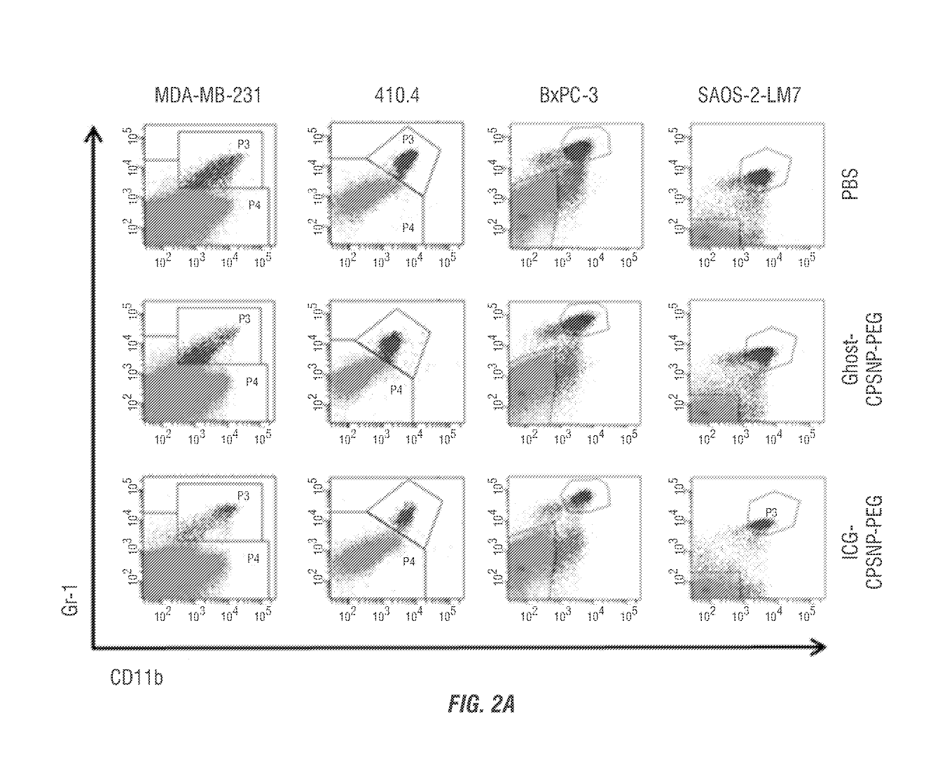

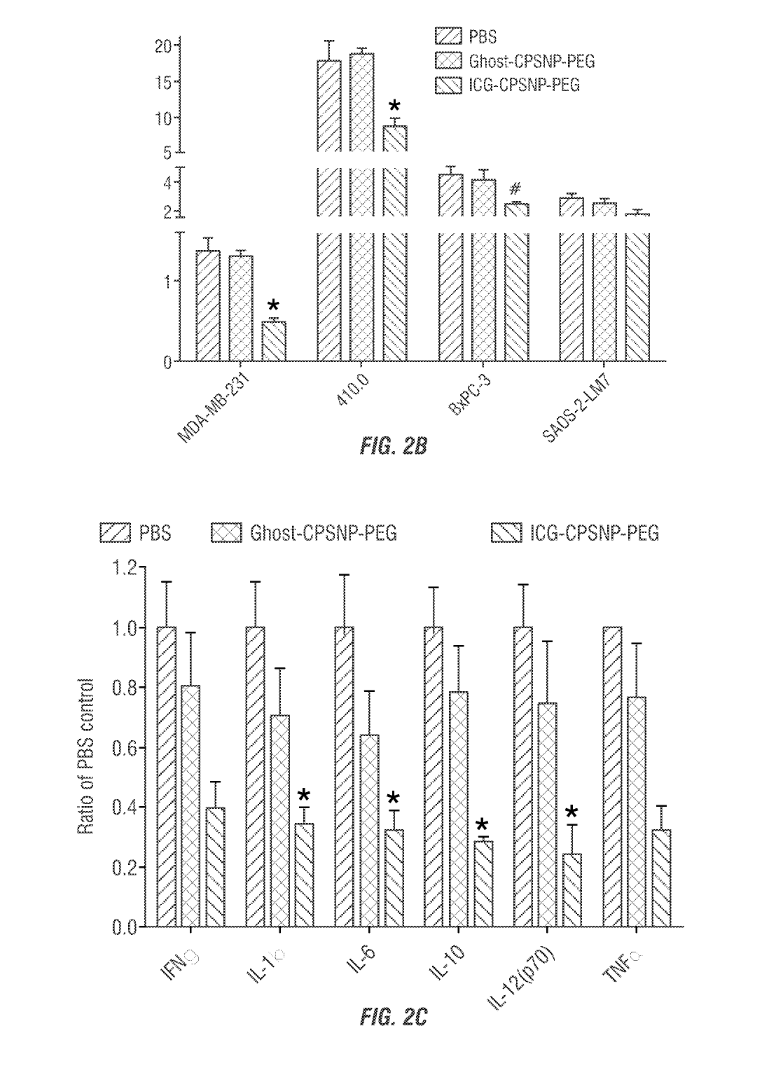

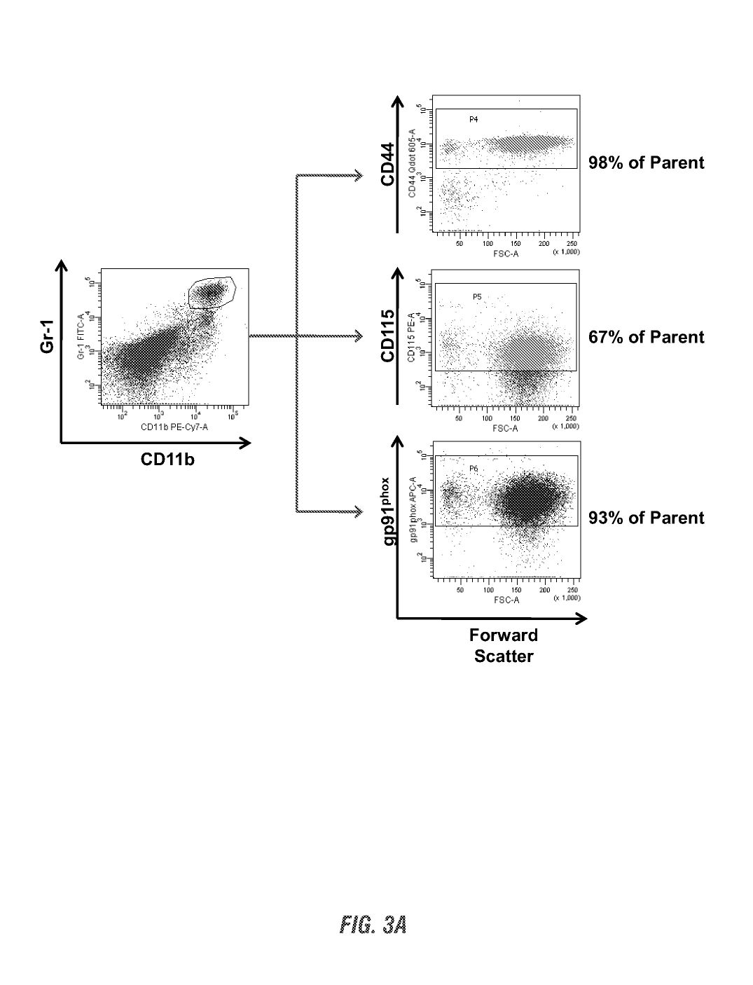

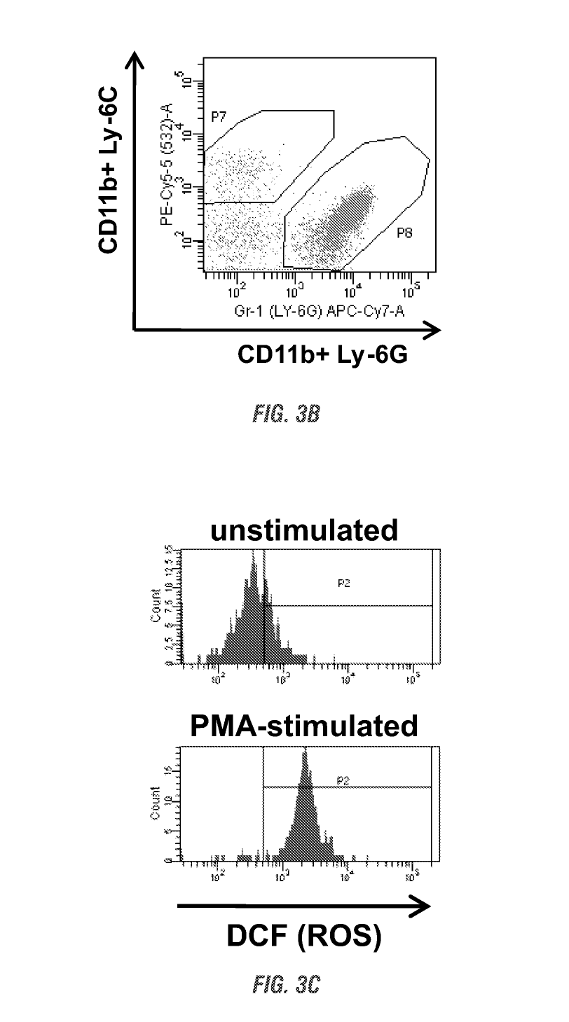

Anticancer T-cell-dependent and -independent immune responses have previously been shown to be negatively regulated by IMCs. To evaluate regulation of IMCs by PhotoImmunoNanoTherapy, MDA-MB-231 or 410.4 tumor-bearing BALB/cJ mice, were sacrificed five days post-NIR laser treatment. All models of tumor-bearing mice contained splenocyte populations of Gr-1+CD11b+IMCs (FIG. 2A). The IMCs of MDA-MB-231 tumor-bearing athymic nude mice also stained positive for the gp91^{phox} subunit of the NADPH oxidase, an enzyme critical to the immunosuppressive nature of MDSCs, and were also predominately CD44+and CD115+, both markers that have been associated with MDSCs (FIG. 3 A-B). As demonstrated using a DCF test for production of reactive oxygen species (ROS), these cells produce ROS when stimulated with phorbol myristate acetate, an indicator which is frequently associated with the immunosuppressive nature of the IMCs (FIG. 3C). The Gr-1+nature of the IMC population in MDA-MB-231 tumor bearing mice was mostly LY-6G+(88%), as opposed to LY-6C (12%), which indicates that this cell population is of a more granulocytic nature. PhotoImmunoNanoTherapy caused a significant decrease in splenic Gr-1+CD11b+IMCs, in MDA-MB-231 tumor-bearing athymic nude mice, whereas treatment with PBS or PEGylated ghost-CPSNPs did not (FIG. 2A). This PhotoImmunoNanoTherapy-induced decrease in splenic IMCs was also observed in 410.4 tumor-bearing BALB/cJ mice (FIG. 2A-B). In a similar manner, PhotoImmunoNanoTherapy caused a significant decrease in splenic IMCs in BxPC-3 orthotopic pancreatic tumor-bearing athymic nude mice and a modest decrease in athymic nude mice bearing SAOS-2-LM7 experimental lung metastases (FIG. 2A-B). An important aspect of IMC, or MDSC, biology is the profound inflammatory milieu which they develop and thrive in. In this study, serum was collected from MDA-MB-231 tumor-bearing athymic nude mice 24 hours following NIR treatment and a cytokine multiplex assay was performed. PhotoImmunoNanoTherapy, but not controls, significantly decreased the levels of IL-1β, IL-6, IL-12, and IL-10, and also appeared to reduce the levels of IFNγ and TNFα although not significantly (FIG. 2C). Combined, these results showed that PhotoImmunoNanoTherapy decreased IMCs and the inflammatory milieu critical to their expansion during tumor progression.

### Example 3

### Immune Effector Cells are Increased by ICG-CPSNP PDT

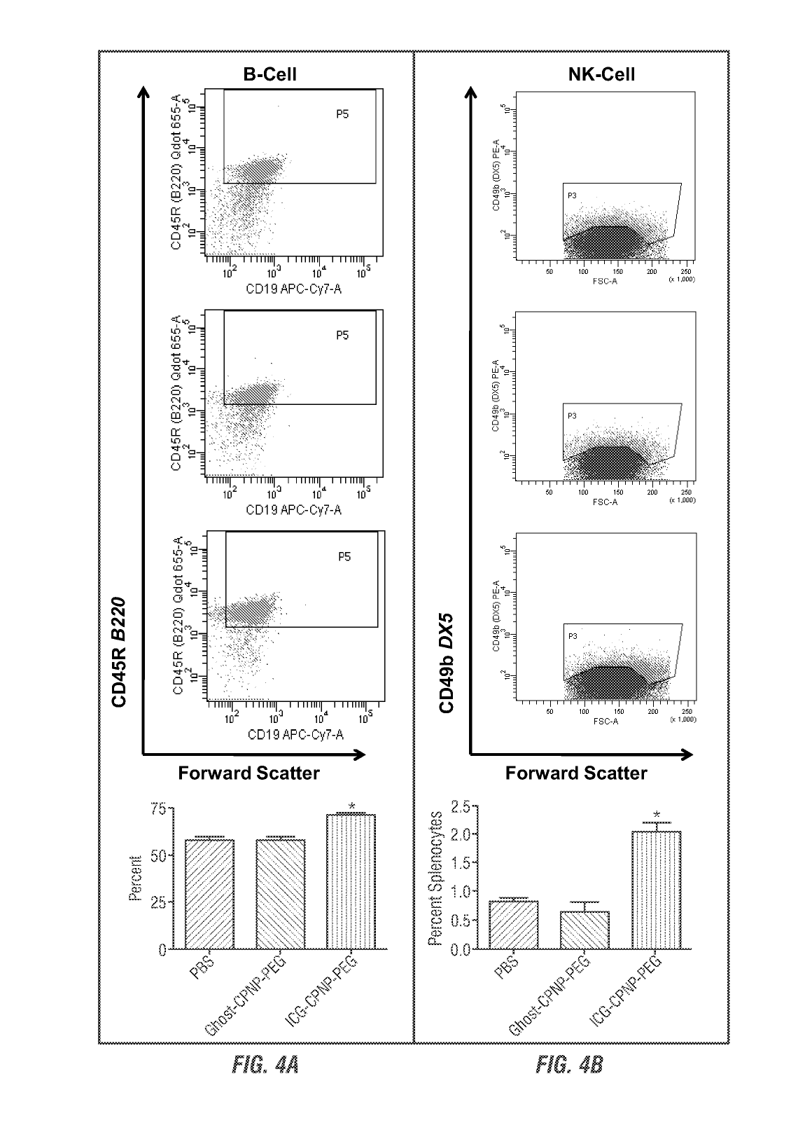

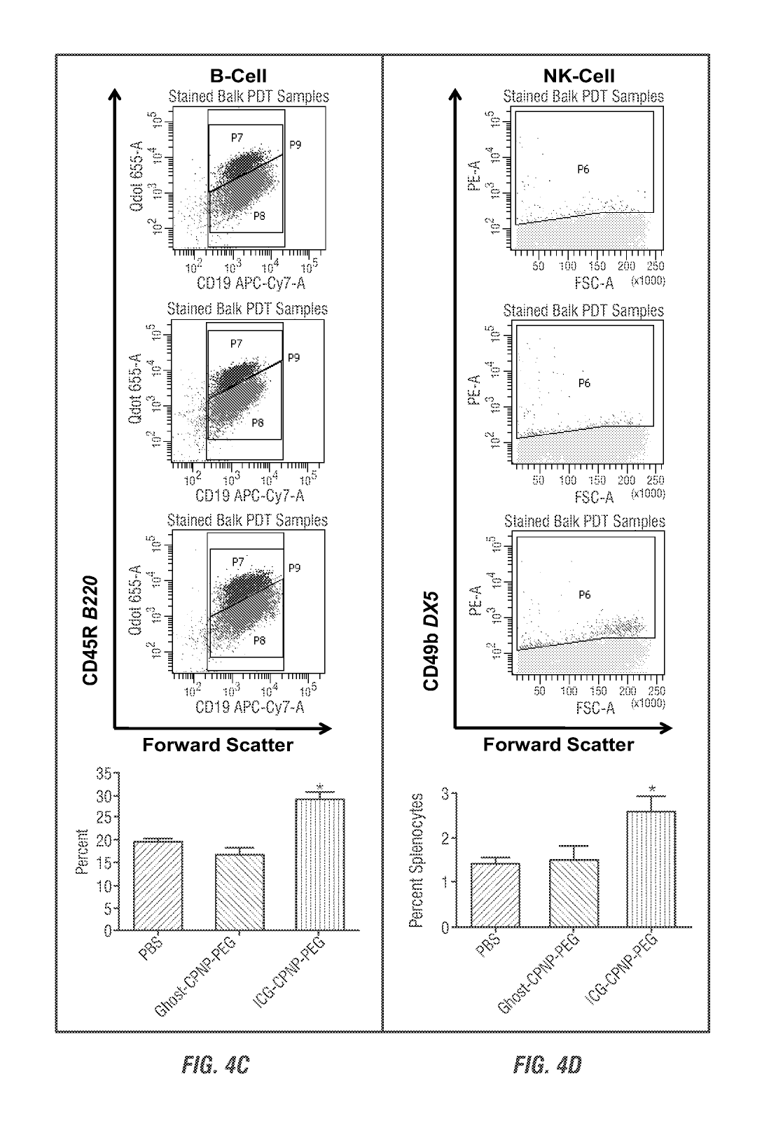

In the absence of an immunosuppressive environment, various immune effector cells have the ability to respond to and attack cancers. As shown above, antitumor efficacy with ICG-CPSNP PDT was observed in both athymic nude mice and Balb/cJ mice, suggesting that T-cell-independent aspects of the immune system were involved in an antitumor immune response, which also downregulated MDSC-like cells. Further evaluation of MDA-MB-231 tumor-bearing athymic nude mice revealed that ICG-CPSNP PDT, but not controls, resulted in a concomitant, statistical increase of splenic B-cells defined as being negative for MDSC markers (Gr-1−CD11b−) and yet CD19+CD45R B220+ (FIG. 4A, left column). Likewise, ICG-CPSNP PDT, but not PBS or photosensitizer-deficient CPSNP controls, caused a significant increase in splenic CD49b DX5+NK cells in MDA-MB-231 tumor-bearing athymic nude mice (FIG. 4A, right column). This observation was notable as the MDSC ability to interfere with NK cells is an important immunosuppressive aspect in athymic nude mice. This ICG-CPSNP PDT-induced increase in splenic NK and B-cells was also observed in 410.4 tumor-bearing Balb/cJ mice (FIG. 4B, left and right columns). Overall, these results showed that ICG-CPSNP PDT diminished MDSC-like cells, while concomitantly stimulating an increase in NK and B-cells in tumor-bearing mice.

### Example 4

### PhotoImmunoNanoTherapy Triggers an Increase of Phosphorylated Bioactive Sphingolipids

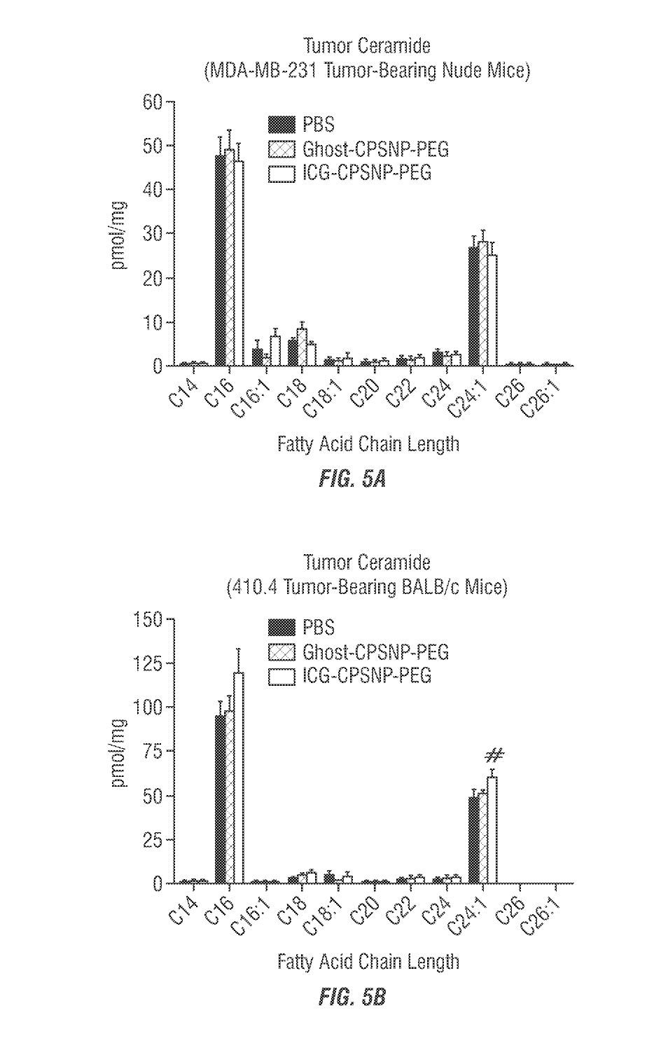

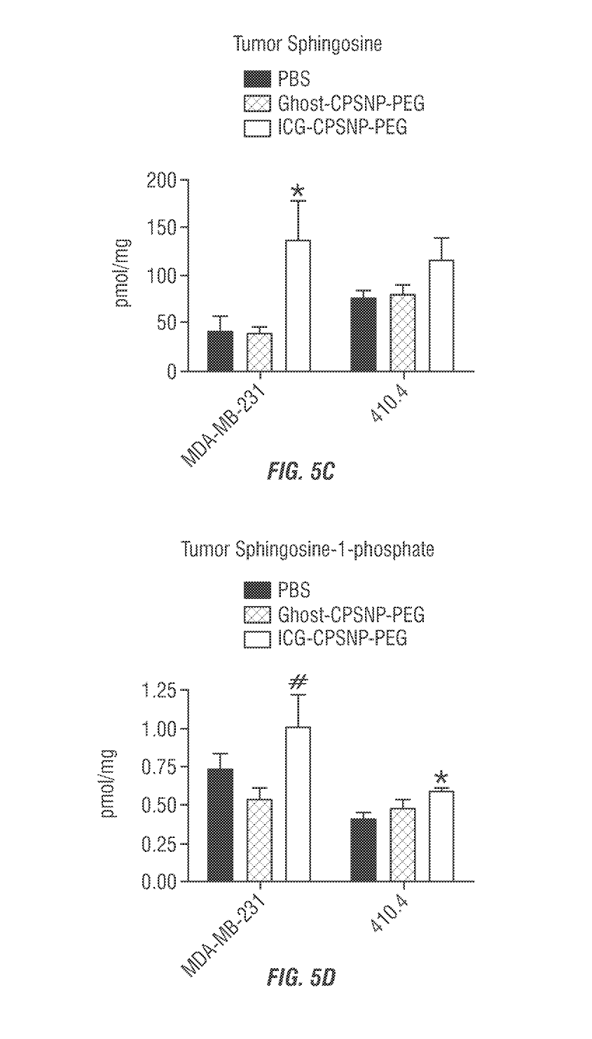

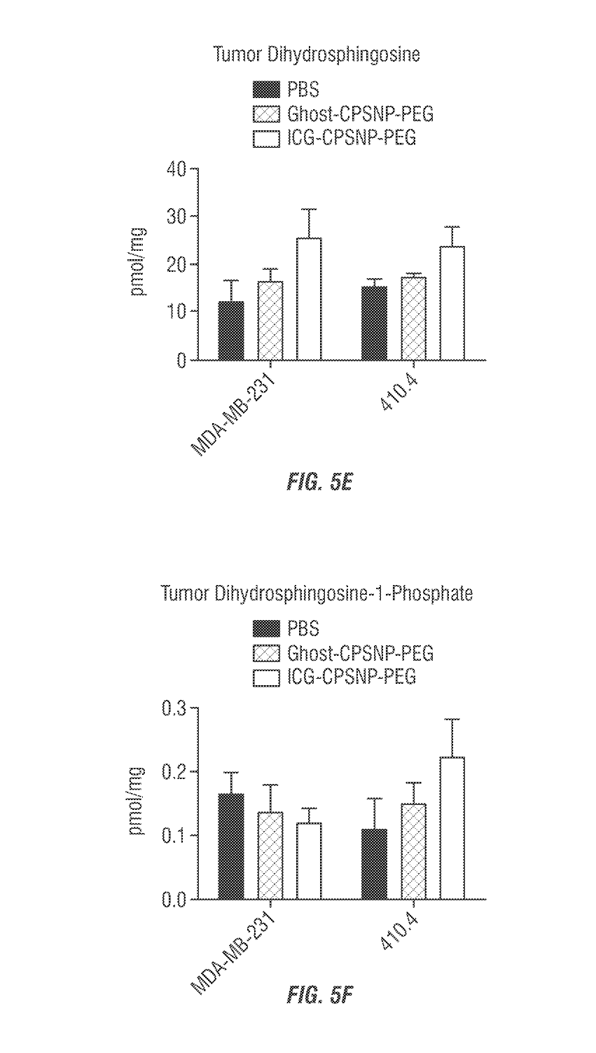

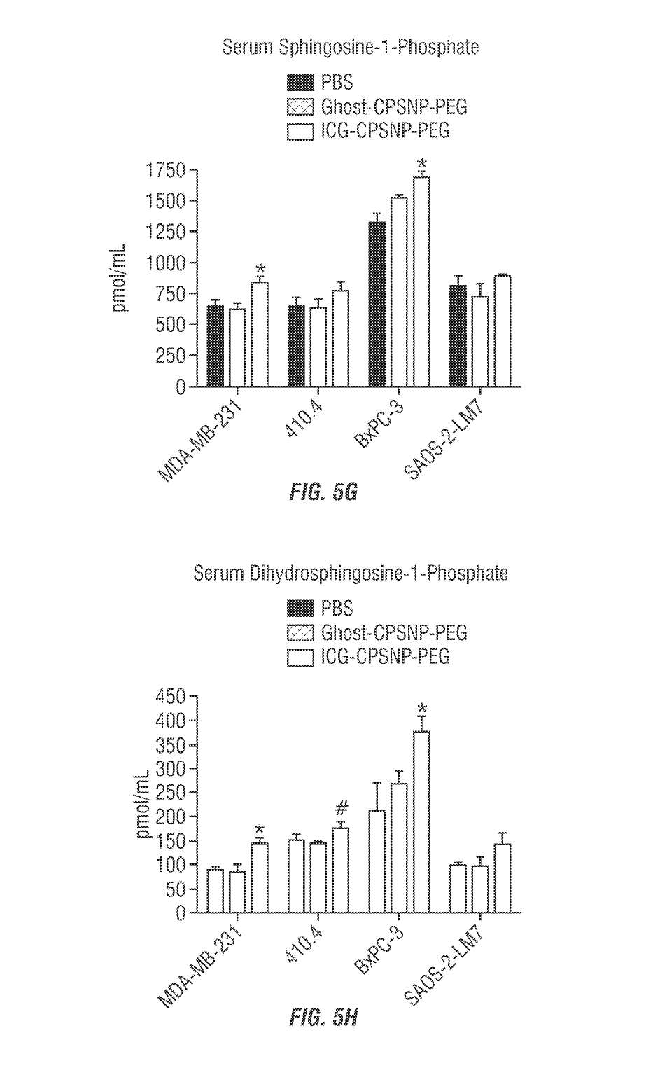

In cancer, sphingolipids such as S1P are often elevated, while ceramides are decreased, providing an environment friendly to tumor growth. Interestingly, levels of tumor and serum ceramides were not affected by ICG-CPSNP PDT (FIG. 5A). It was therefore hypothesized that the molecular mechanism mediating ICG-CPSNP PDT may involve phosphorylated sphingolipid metabolites. The commercial production of sphingolipids is well known in the art.

To explore how PhotoImmunoNanoTherapy could be regulating the immune system, an analysis of the “sphingolipidome” was studied in tumors and serum collected from treated tumor-bearing mice. As PhotoImmunoNanoTherapy modulated the immune system, and was efficacious in both athymic nude mice and BALB/cJ mice, in depth “sphingolipidomic” studies were performed in athymic nude mice bearing MDA-MB-231 tumors to focus more precisely on mediation of T-cell-independent immunity as well as BALB/cJ mice bearing 410.4 tumors (FIG. 5A-F). Tumor sphingolipidomic studies revealed that ceramides were mostly unchanged with the exception of a minor increase in C24:1 in BALB/cJ mice (410.4 tumors) (FIG. 5B). Intriguingly, an increase in tumor S1P was observed as a function of PhotoImmunoNanoTherapy in both models (FIG. 5D), as well as an increase in the precursor sphingosine in the athymic nude mouse model (MDA-MB-231 tumor) (FIG. 5C). In contrast, a sphingolipidomic analysis of the serum of treated mice revealed that both S1P and its related bioactive sphingolipid dihydrosphingosine-1-phosphate (dhS1P) were significantly elevated in the serum of PhotoImmunoNanoTherapy-treated athymic nude mice with subcutaneous MDA-MB-231 tumors or with orthotopic BxPC-3 tumors (FIG. 5G-H). Modest elevations of serum dhS1P were also observed in the serum of PhotoImmunoNanoTherapy-treated BALB/cJ mice bearing 410.4 tumors (FIG. 5H). Of particular interest, the mass levels of phosphorylated sphingolipid species were much higher in serum than in tumor tissue possibly reflective of a release of phosphorylated sphingolipids in response to PhotoImmunoNanoTherapy. Intriguingly, the increase in the amount of dhS1P was more dramatic than the increase in S1P. In the MDA-MB-231, BxPC-3, and SAOS-2-LM7 models there were a 65%, 79%, and 43% increase in dhS1P, respectively, and these compared with respective increases in S1P of only 29%, 27%, and 10%. These data suggest that PhotoImmunoNanoTherapy initiates specific alterations of the “sphingolipidome”, possibly resulting in the production and release of bioactive phosphorylated sphingolipid metabolites into systemic circulation. Like S1P, dhS1P is generated by sphingosine kinase (SphK) activity, but unlike S1P, no significant role has been attributed to dhS1P. Much attention has been given to the role of ceramides in the induction of cell death, and in particular in response to chemotherapy, radiation therapy, and even PDT. In cancer, sphingolipids such as S1P are often elevated, while ceramides are decreased, providing an environment friendly to tumor growth. Therefore, the specific increase in S1P and dhS1P observed in response to Photo ImmunoNanoTherapy was particularly intriguing and thought to mediate a potentially novel antitumor mechanism.

### Example 5

### Sphingosine Kinase 2 Mediates the Antitumor Effects of PhotoImmunoNanoTherapy

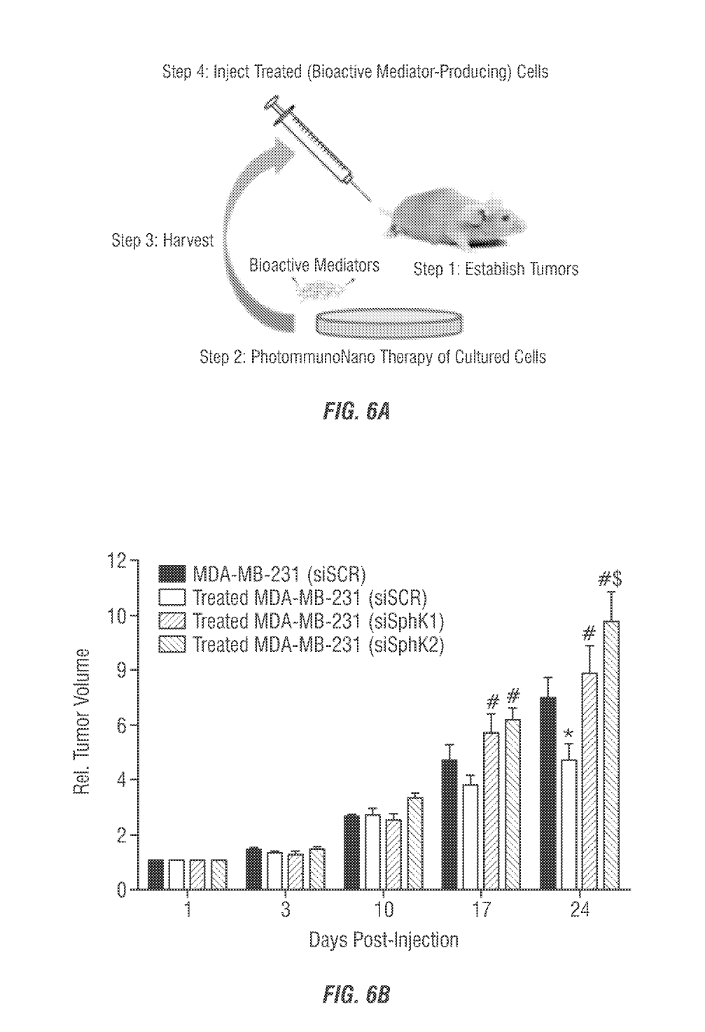

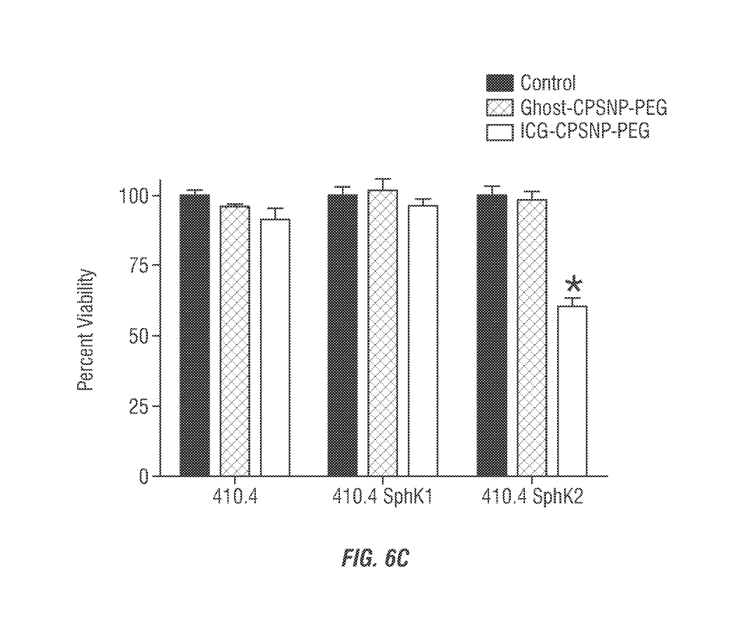

To confirm a potentially novel role for SphK and S1P and/or dhS1P in modulating the antitumor effect of PhotoImmunoNanoTherapy, an experimental model was developed where MDA-MB-231 cells were treated in culture with PhotoImmunoNanoTherapy, and then injected systemically into tumor-bearing mice (FIG. 6A). The premise was that the PhotoImmunoNanoTherapy treatment would trigger the release of S1P, dhS1P, or other S1P/dhS1P-regulated bioactive mediators, and that this would exert an antitumor effect.

Indeed, this experimental strategy blocked tumor growth, while abrogation of SphK1 or SphK2 with siRNA completely eliminated any antitumor effect (FIG. 6B). These findings demonstrated that lipids generated by SphK in cancer cells mediate the antitumor effect of PhotoImmunoNanoTherapy.

To verify the role of SphKs, 410.4 cells stably expressing either SphK1 or SphK2 were exposed to normally non-toxic PhotoImmunoNanoTherapy conditions. Only SphK2 expressing cells were significantly sensitive (FIG. 6C), further implicating SphK2 as the key regulator of PhotoImmunoNanoTherapy. Intriguingly, it has been reported that S1P generated in the nucleus by SphK2 is implicated in epigenetic regulation, and it is possible that multiple phosphorylated lipid signaling molecules mediate the efficacy of PhotoImmunoNanoTherapy through effects at surface receptors or as epigenetic regulators. Indeed, nuclear production of S1P by SphK2 was recently shown to mediate epigenetic regulation of genes governing cellular stress. In the present study, SphK2 was shown to mediate the efficacy of PhotoImmunoNanoTherapy perhaps due to epigenetic regulation of an anti-inflammatory program that may subsequently be responsible for the observed decrease in tumor-associated inflammation and IMCs. It is also noteworthy that the study evaluating the epigenetic role for S1P in the nucleus also detected dhS1P and never distinguished a specific role for either lipid. Moreover, the diverse membrane localization of SphK2 puts it in an optimal subcellular position to generate dhS1P at membranes that are rich in dihydrosphingosine, such as the endoplasmic reticulum.

### Example 6

### Impact of dhS1P on MDSC Cell Surface Markers

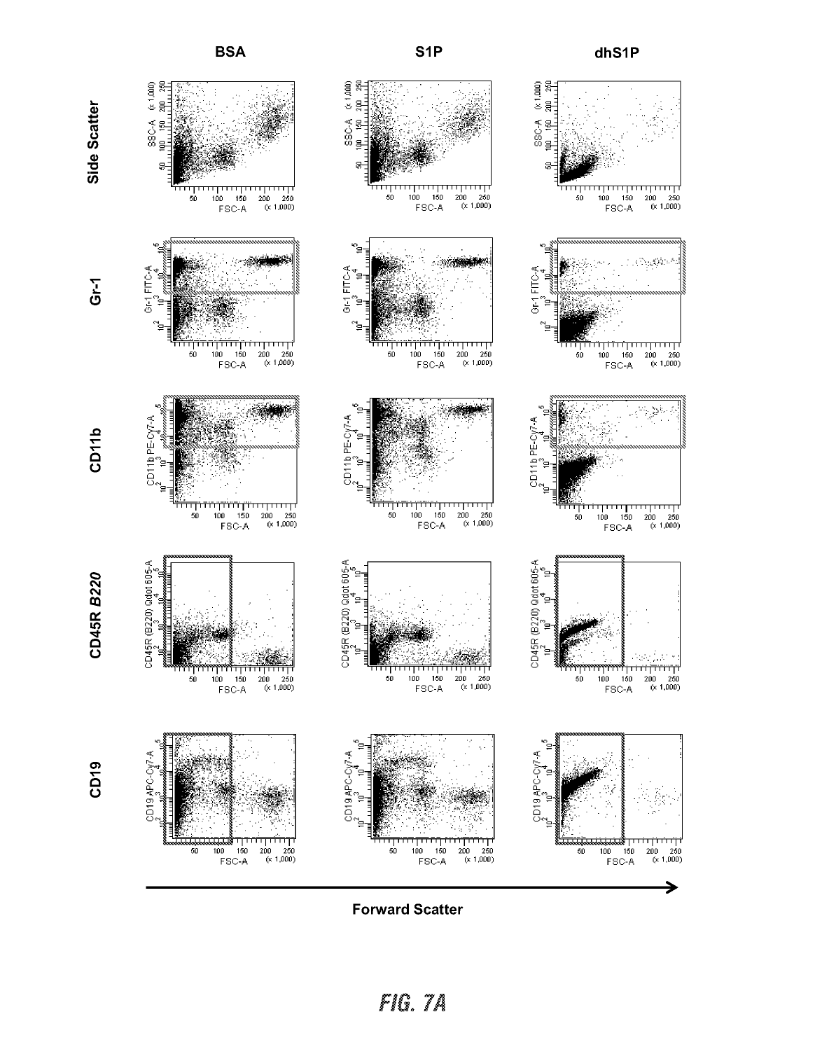

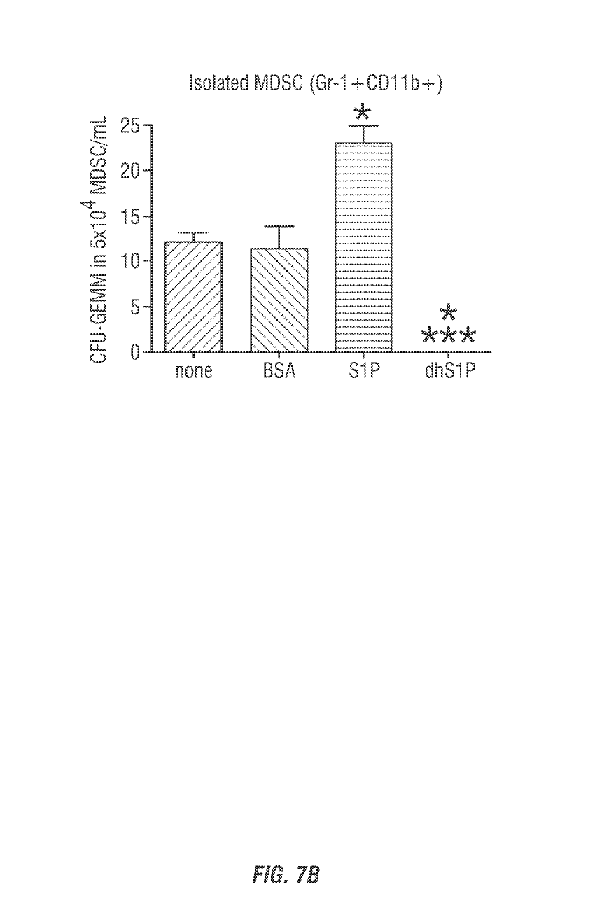

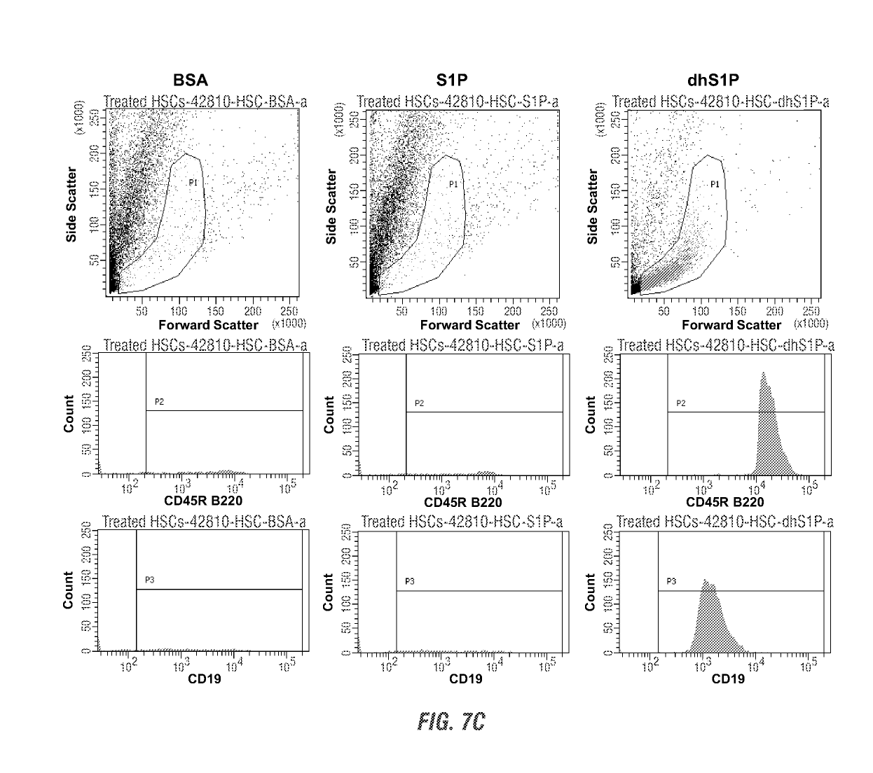

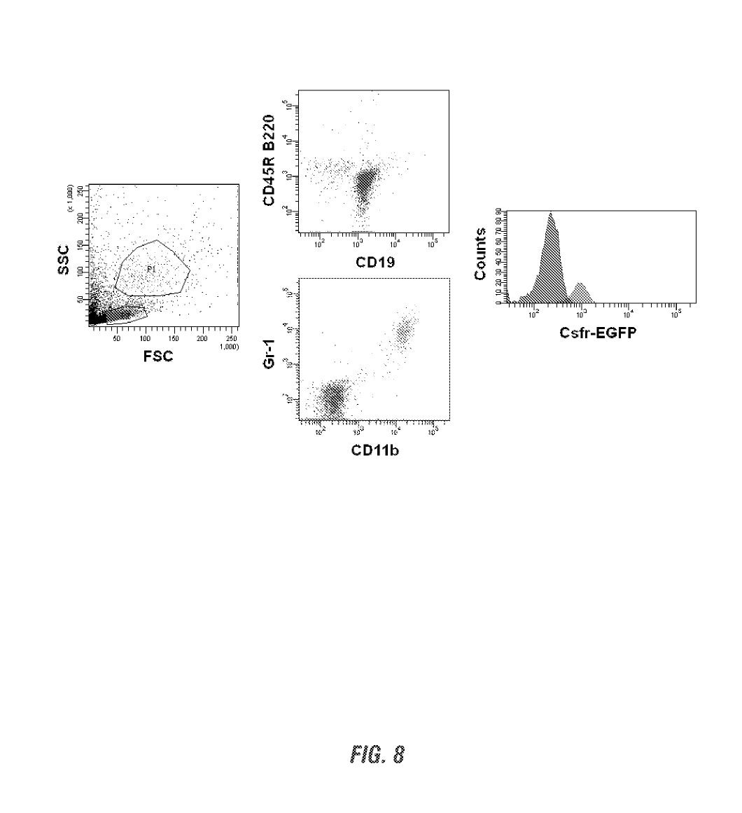

The effect of dhS1P was further investigated at the level of MDSC-like cells, which were reduced as a function of treatment. The effects of dhS1P were directly compared with those of SIP as to delineate a difference in their physiological roles. Tumor-expanded IMCs/MDSCs were isolated and exposed in culture to either dhS1P or S1P. The comparison demonstrated that only dhS1P exerted an effect on isolated IMCs/MDSCs in culture. Specifically, multicolor flow cytometry revealed that cells bearing the surface characteristics of IMCs/MDSCs were completely ablated under normal culture conditions by dhS1P treatment, but not S1P treatment (FIG. 7A). This was confirmed by repeating the same dhS1P, or S1P, treatments on isolated IMCs but in growth factor-supplemented media as a colony forming assay. Isolated IMCs were cultured in CFU (colony forming unit)-GEMM (granulocyte, erythrocyte, monocyte, megakaryocyte) supportive semi-solid media and formed GEMM colonies indicative of their multipotent myeloid progenitor nature (FIG. 7B). This specific colony growth was shown to be dramatically augmented by S1P treatment. In contrast, CFU-GEMM colony formation was completely abrogated by exposure to dhS1P, indicative of the lipid's potent regulatory effect. Intriguingly, dhS1P exposure also promoted the expansion of a new population of cells in culture which displayed CD19 and CD45R B220 on their surface (FIG. 7A). It is possible that this effect is indirect, in that dhS1P-mediated suppression of IMCs/MDSCs simply removes a blockade of lymphoid differentiation. In agreement with this idea, dhS1P mediated the expansion of the same CD19+CD45R B220+ cellular population from isolated hematopoietic progenitors was observed (FIG. 7A). Separately performed lineage tracing analysis confirmed that this population is not of myeloid origin (FIG. 8), and this suggested that the perceived expansion of B-cells from isolated IMCs was simply due to the presence of a “contaminating” progenitor. This conclusion is further likely considering the purity obtained with the high-speed cell sorter used to isolate IMCs/MDSCs was between 85-95%.

To more closely evaluate the genetic consequences of the dhS1P-induced decrease in isolated MDSC-like cells and the increase in cells bearing the surface markers of B-cells, a RNA microarray analysis was conducted. MDSC-like cells were isolated from MDA-MB-231 tumor-bearing athymic nude mice, exposed for 24 hours to dhS1P or vehicle (BSA), followed by RNA extraction, and a whole-genome microarray was performed. As compared to vehicle-treated MDSC-like cells, dhS1P treatment of isolated

MDSC-like cells altered the expression of a variety of genes. Using an unpaired t-test, a fold-change cut-off of 1.2, and a p-value cut-off of 0.05, 319 significantly regulated genes were observed (Table 1). Analysis of these regulated genes using Ingenuity Pathway Analysis (Ingenuity Systems, Redwood City, Calif.) revealed relevance to several networks of gene products, the top three networks of which were linked to hematological system development and function, cellular growth and proliferation, as well as cell to cell signaling. A closer inspection of the microarray data revealed several interesting myeloid cell-linked genes, which were downregulated, including Clec4e, Cxcr2, and Pilra. Likewise, several interesting upregulated genes associated with B-cells were noted, including Lgals1, Ly6d, and Vpreb3. These observations were consistent with the flow cytometry analysis which showed that dhS1P induced a decrease in MDSC-like cells and an increase in B-cells. Altogether, the microarray data supported the flow cytometry data, further demonstrating that dhS1P initiated changes in isolated MDSC-like cells consistent with their decrease and an emergence of a new population of B-cells, likely from hematopoietic progenitors.

TABLE 1
Significantly regulated genes following dhS1P treatment of isolated MDSCs

| Symbol | Accession | Regulation | Description |
|---|---|---|---|
| Ankrd49 | NM_019683.2 | down | ankyrin repeat domain 49 |
| Aqp9 | NM_022026.2 | down | aquaporin 9 |
| Arl2bp | NM_024269.2 | down | ADP-ribosylation factor-like 2 |
| | | | binding protein |
| Arrdc4 | NM_025549.1 | down | arrestin domain containing 4 |
| Asf1a | NM_025541.2 | down | ASF1 anti-silencing function 1 |
| | | | homolog A (S. cerevisiae) |
| Ash1l | NM_138679.2 | down | ash1 (absent, small, or homeotic)-like |
| | | | (Drosophila) |
| Atm | NM_007499.1 | down | ataxia telangiectasia mutated |
| | | | homolog (human) |
| Bmi1 | NM_007552.3 | down | Bmi1 polycomb ring finger oncogene |
| Ccdc125 | NM_183115.1 | down | coiled-coil domain containing 125 |
| Ccnd2 | NM_009829 | down | cyclin D2 |
| Cd14 | NM_009841.2 | down | CD14 antigen |
| Cep68 | NM_172260.1 | down | centrosomal protein 68 |
| Chm | NM_018818.2 | down | choroidermia |
| Clcn3 | NM_173876.1 | down | chloride channel 3 |
| Clec4e | NM_019948.1 | down | C-type lectin domain family 4, |
| | | | member e |
| Cmah | NM_007717.1 | down | cytidine monophospho-N- |
| | | | acetylneuraminic acid hydroxylase |
| Cnot4 | NM_016877 | down | CCR4-NOT transcription complex, |
| | | | subunit 4 |
| Cobll1 | NM_177025.3 | down | Cobl-like 1 |
| Cpd | NM_007754.1 | down | carboxypeptidase D |
| Crbn | NM_021449.1 | down | cereblon |
| Cxcr2 | NM_009909.2 | down | chemokine (C-X-C motif) receptor 2 |
| Cyfip1 | NM_011370.1 | down | cytoplasmic FMR1 interacting |
| | | | protein 1 |
| Cyp51 | NM_020010 | down | cytochrome P450, family 51 |
| Ddx6 | NM_181324.2 | down | DEAD (Asp-Glu-Ala-Asp) box |
| | | | polypeptide 6 |
| Ddx6 | NM_007841.2 | down | DEAD (Asp-Glu-Ala-Asp) box |
| | | | polypeptide 6 |
| Dhrs9 | NM_175512.2 | down | dehydrogenase/reductase (SDR |
| | | | family) member 9 |
| Dusp6 | NM_026268.1 | down | dual specificity phosphatase 6 |
| Dync1li1 | NM_146229.1 | down | dynein cytoplasmic 1 light |
| | | | intermediate chain 1 |
| Edaradd | NM_133643 | down | EDAR (ectodysplasin-A receptor)- |
| | | | associated death domain |
| Egr2 | NM_010118.1 | down | early growth response 2 |
| Eif5 | NM_173363.2 | down | eukaryotic translation initiation factor 5 |
| Enpp4 | NM_199016.1 | down | ectonucleotide |
| | | | pyrophosphatase/phosphodiesterase 4 |
| Eprs | NM_029735.1 | down | glutamyl-prolyl-tRNA synthetase |
| F13a1 | NM_028784.2 | down | coagulation factor XIII, A1 subunit |
| F2r | NM_010169.2 | down | coagulation factor II (thrombin) |
| | | | receptor |
| Fam63b | NM_172772.1 | down | family with sequence similarity 63, |
| | | | member B |
| Fam65b | NM_178658.2 | down | family with sequence similarity 65, |
| | | | member B |
| Fam76a | NM_145553.1 | down | family with sequence similarity 76, |
| | | | member A |
| Fas | NM_007987.1 | down | Fas (TNF receptor superfamily |
| | | | member 6) |
| Fbxl5 | NM_178729.2 | down | F-box and leucine-rich repeat protein 5 |
| Fli1 | NM_008026 | down | Friend leukemia integration 1 |
| Fnbp4 | NM_018828.1 | down | formin binding protein 4 |
| Foxp1 | NM_053202.1 | down | forkhead box P1 |
| Fyb | NM_011815.1 | down | FYN binding protein |
| Gatad2b | NM_139304 | down | GATA zinc finger domain containing |
| | | | 2B |
| Git2 | NM_019834.2 | down | G protein-coupled receptor kinase- |
| | | | interactor 2 |
| Gna13 | NM_010303.2 | down | guanine nucleotide binding protein, |
| | | | alpha 13 |
| Golga2 | NM_133852.1 | down | golgi autoantigen, golgin subfamily a, 2 |
| Gp1ba | NM_010326.1 | down | glycoprotein 1b, alpha polypeptide |
| Gp5 | NM_008148.2 | down | glycoprotein 5 (platelet) |
| Gpd2 | NM_010274.2 | down | glycerol phosphate dehydrogenase 2, |
| | | | mitochondrial |
| Hcls1 | NM_008225.1 | down | hematopoietic cell specific Lyn |
| | | | substrate 1 |
| Hdac4 | NM_207225.1 | down | histone deacetylase 4 |
| Herpud2 | NM_020586.1 | down | HERPUD family member 2 |
| Hif1a | NM_010431.1 | down | hypoxia inducible factor 1, alpha |
| | | | subunit |
| Hist1h2bg | NM_178196.2 | down | histone cluster 1, H2bg |
| Hist1h2bh | NM_178197.1 | down | histone cluster 1, H2bh |
| Hist1h3a | NM_013550.3 | down | Hist1h3a histone cluster 1, H3a |
| Il28ra | NM_174851.2 | down | interleukin 28 receptor alpha |
| Inhba | NM_008380.1 | down | inhibin beta-A |
| Itgav | NM_008402.1 | down | integrin alpha V |
| Kdsr | NM_027534.1 | down | 3-ketodihydrosphingosine reductase |
| Khdrbs1 | NM_011317.2 | down | KH domain containing, RNA |
| | | | binding, signal transduction |
| | | | associated 1 |
| Klf7 | NM_033563 | down | Kruppel-like factor 7 (ubiquitous) |
| Larp4b | NM_172585.1 | down | La ribonucleoprotein domain family, |
| | | | member 4B |
| Lcp1 | NM_008879.2 | down | lymphocyte cytosolic protein 1 |
| Lpcat2 | NM_173014.1 | down | lysophosphatidylcholine |
| | | | acyltransferase 2 |
| Mat2a | NM_145569 | down | methionine adenosyltransferase II, |
| | | | alpha |
| Mbd4 | NM_010774.1 | down | methyl-CpG binding domain protein 4 |
| Mef2c | NM_025282 | down | myocyte enhancer factor 2C |
| Mitf | NM_008601 | down | microphthalmia-associated |
| | | | transcription factor |
| Mobkl1b | NM_145571 | down | MOB1, Mps One Binder kinase |
| | | | activator-like 1B (yeast) |
| Mpp5 | NM_019579.1 | down | membrane protein, palmitoylated 5 |
| | | | (MAGUK p55 subfamily member 5) |
| Mrpl9 | NM_030116.1 | down | mitochondrial ribosomal protein L9 |
| Mrvi1 | NM_194464 | down | MRV integration site 1 |
| Nab1 | NM_008667.2 | down | Ngfi-A binding protein 1 |
| Nfatc3 | NM_010901 | down | nuclear factor of activated T-cells, |
| | | | cytoplasmic, calcineurin-dependent 3 |
| Nfe212 | NM_010902.2 | down | nuclear factor, erythroid derived 2, |
| | | | like 2 |
| Nop58 | NM_018868 | down | NOP58 ribonucleoprotein homolog |
| | | | (yeast) |
| Numb | NM_010949.1 | down | numb gene homolog (Drosophila) |
| Olfm4 | NM_001030294.1 | down | olfactomedin 4 |
| Olfr455 | NM_001081301.1 | down | olfactory receptor 455 |
| Opa3 | NM_207525.1 | down | optic atrophy 3 (human) |
| P2ry13 | NM_028808.1 | down | purinergic receptor P2Y, G-protein |
| | | | coupled 13 |
| Papola | NM_011112 | down | poly (A) polymerase alpha |
| Pdcl | NM_026176.2 | down | phosducin-like |
| Pdpk1 | NM_001080773.1 | down | 3-phosphoinositide dependent protein |
| | | | kinase-1 |
| Phf7 | NM_027949.1 | down | PHD finger protein 7 |
| Pias4 | NM_021501.1 | down | protein inhibitor of activated STAT 4 |
| Pik3ap1 | NM_031376.1 | down | phosphoinositide-3-kinase adaptor |
| | | | protein 1 |
| Pik3cg | NM_020272 | down | phosphoinositide-3-kinase, catalytic, |
| | | | gamma polypeptide |
| Pilra | NM_153510.1 | down | paired immunoglobin-like type 2 |
| | | | receptor alpha |
| Pira11 | NM_011088.1 | down | paired-Ig-like receptor A11 |
| Pira6 | NM_008848.1 | down | paired-Ig-like receptor A6 |
| Pja2 | NM_144859 | down | praja 2, RING-H2 motif containing |
| Pkn2 | NM_178654 | down | protein kinase N2 |
| Ppbp | NM_023785.1 | down | Ppbp pro-platelet basic protein |
| Ppp1cb | NM_172707 | down | protein phosphatase 1, catalytic |
| | | | subunit, beta isoform |
| Prmt5 | NM_013768 | down | protein arginine N-methyltransferase 5 |
| Ptp4a2 | NM_008974.2 | down | protein tyrosine phosphatase 4a2 |
| Ptprc | NM_011210.1 | down | protein tyrosine phosphatase, receptor |
| | | | type, C |
| Ralgds | NM_009058.1 | down | ral guanine nucleotide dissociation |
| | | | stimulator |
| Ranbp6 | NM_177721.2 | down | RAN binding protein 6 |
| Rasa2 | NM_053268 | down | RAS p21 protein activator 2 |
| Rbl2 | NM_011250 | down | retinoblastoma-like 2 |
| Rbm39 | NM_133242.1 | down | RNA binding motif protein 39 |
| Rbms1 | NM_020296 | down | RNA binding motif, single stranded |
| | | | interacting protein 1 |
| Rnf4 | NM_011278.1 | down | ring finger protein 4 |
| Rock1 | NM_009071 | down | Rho-associated coiled-coil containing |
| | | | protein kinase 1 |
| Rsf1 | NM_001081267.1 | down | remodeling and spacing factor 1 |
| Sdf4 | NM_011341.3 | down | stromal cell derived factor 4 |
| Sdpr | NM_138741.1 | down | serum deprivation response |
| Senp7 | NM_001003972.1 | down | SUMO1/sentrin specific peptidase 7 |
| Serpinb2 | NM_011111.2 | down | serine (or cysteine) peptidase |
| | | | inhibitor, clade B, member 2 |
| Sgms1 | NM_144792.2 | down | sphingomyelin synthase 1 |
| Sirpb1a | NM_001002898.1 | down | signal-regulatory protein beta 1A |
| Skp2 | NM_013787.1 | down | S-phase kinase-associated protein 2 |
| | | | (p45) |
| Smek2 | NM_134034 | down | SMEK homolog 2, suppressor of |
| | | | mek1 (Dictyostelium) |
| Srrm4 | NM_026886.1 | down | serine/arginine repetitive matrix 4 |
| Stard4 | NM_133774 | down | StAR-related lipid transfer (START) |
| | | | domain containing 4 |
| Stk3 | NM_019635.2 | down | serine/threonine kinase 3 (Ste20, |
| | | | yeast homolog) |
| Tbl1x | NM_020601 | down | transducin (beta)-like 1 X-linked |
| Tes | NM_011570.2 | down | testis derived transcript |
| Tex9 | NM_009359.2 | down | testis expressed gene 9 |
| Tgs1 | NM_054089.2 | down | trimethylguanosine synthase homolog |
| | | | (S. cerevisiae) |
| Tmem108 | NM_178638.2 | down | transmembrane protein 108 |
| Tnip1 | NM_021327.1 | down | TNFAIP3 interacting protein 1 |
| Tob2 | NM_020507.2 | down | transducer of ERBB2, 2 |
| Top1 | NM_009408.1 | down | topoisomerase (DNA) I |
| Tpk1 | NM_013861 | down | thiamine pyrophosphokinase |
| Traf2 | NM_009422.1 | down | TNF receptor-associated factor 2 |
| Txndc11 | NM_029582.1 | down | thioredoxin domain containing 11 |
| Usp7 | NM_001003918.1 | down | ubiquitin specific peptidase 7 |
| Vwf | NM_011708.2 | down | Von Willebrand factor homolog |
| Was | NM_009515.1 | down | Wiskott-Aldrich syndrome homolog |
| | | | (human) |
| Wdr37 | NM_172445.1 | down | WD repeat domain 37 |
| Xpnpep3 | NM_177310 | down | X-prolyl aminopeptidase |
| | | | (aminopeptidase P) 3, putative |
| Zdhhc21 | NM_026647.2 | down | zinc finger, DHHC domain |
| | | | containing 21 |
| Zeb2 | NM_015753.2 | down | zinc finger E-box binding homeobox 2 |
| Zfp106 | NM_011743 | down | zinc finger protein 106 |
| Zfp131 | NM_028245.1 | down | zinc finger protein 131 |
| Zfp292 | NM_013889.1 | down | zinc finger protein 292 |
| Zfp318 | NM_207671.2 | down | zinc finger protein 318 |
| Zfp516 | NM_183033 | down | zinc finger protein 516 |
| Zmat1 | NM_175446.2 | down | zinc finger, matrin type 1 |
| Zmynd8 | NM_027230 | down | zinc finger, MYND-type containing 8 |
| 1600002K0 | NM_027207.1 | up | RIKEN cDNA 1600002K03 gene |
| 3Rik | | | |
| 1700030K0 | NM_028170.1 | up | RIKEN cDNA 1700030K09 gene |
| 9Rik | | | |
| 2010002N0 | NM_134133.1 | up | RIKEN cDNA 2010002N04 gene |
| 4Rik | | | |
| 2310007A1 | NM_025506 | up | RIKEN cDNA 2310007A19Rik |
| 9Rik | | | |
| 2510012J0 | NM_027381.1 | up | RIKEN cDNA 2510012J08 gene |
| 8Rik | | | |
| 2900010M | NM_026063.1 | up | RIKEN cDNA 2900010M23 gene |
| 23Rik | | | |
| 3110056O0 | NM_175195.2 | up | RIKEN cDNA 3110056O03 gene |
| 3Rik | | | |
| 5430435G2 | NM_145509.1 | up | RIKEN cDNA 5430435G22 gene |
| 2Rik | | | |
| 9130011E1 | NM_198296.1 | up | RIKEN cDNA 9130011E15 gene |
| 5Rik | | | |
| 9430023L2 | NM_026566.1 | up | RIKEN cDNA 9430023L20 gene |
| 0Rik | | | |
| Abi3 | NM_025659 | up | ABI gene family, member 3 |
| Afg3l1 | NM_054070.1 | up | AFG3(ATPase family gene 3)-like 1 |
| | | | (yeast) |
| Ahnak2 | NM_001033476.1 | up | AHNAK nucleoprotein 2 |
| Ahsa1 | NM_146036.1 | up | AHA1, activator of heat shock |
| | | | protein ATPase homolog 1 (yeast) |
| Aif1 | NM_019467.2 | up | allograft inflammatory factor 1 |
| Akap8l | NM_017476.1 | up | A kinase (PRKA) anchor protein 8- |
| | | | like |
| Akr1b3 | NM_009658 | up | aldo-keto reductase family 1, member |
| | | | B3 (aldose reductase) |
| Anapc5 | NM_021505.1 | up | anaphase-promoting complex subunit 5 |
| Anp32e | NM_023210.2 | up | acidic (leucine-rich) nuclear |
| | | | phosphoprotein 32 family, member E |
| Anpep | NM_008486.1 | up | alanyl (membrane) aminopeptidase |
| Appl2 | NM_145220.1 | up | adaptor protein, phosphotyrosine |
| | | | interaction, PH domain and leucine |
| | | | zipper containing 2 |
| Atad3a | NM_179203.1 | up | ATPase family, AAA domain |
| | | | containing 3A |
| Atf5 | NM_030693.1 | up | activating transcription factor 5 |
| Atp13a2 | NM_029097.1 | up | ATPase type 13A2 |
| Atp2a2 | NM_009722.1 | up | ATPase, Ca++ transporting, cardiac |
| | | | muscle, slow twitch 2 |
| Atp6v1g2 | NM_023179.2 | up | ATPase, H+ transporting, lysosomal |
| | | | V1 subunit G2 |
| Atpif1 | NM_007512.2 | up | ATPase inhibitory factor 1 |
| Bax | NM_007527.2 | up | BCL2-associated X protein |
| Bicd2 | NM_001039180.1 | up | bicaudal D homolog 2 (Drosophila) |
| Blvra | NM_026678.3 | up | biliverdin reductase A |
| Car13 | NM_024495.2 | up | carbonic anhydrase 13 |
| Ccdc107 | NM_001037913.1 | up | coiled-coil domain containing 107 |
| Cd55 | NM_010016.1 | up | CD55 antigen |
| Cd59a | NM_007652.2 | up | CD59a antigen |
| Cd63 | NM_007653.1 | up | CD63 antigen |
| Cdk5rap3 | NM_030248.1 | up | CDK5 regulatory subunit associated |
| | | | protein 3 |
| Cenpb | NM_007682.2 | up | centromere protein B |
| Cfb | NM_008198.1 | up | complement factor B |
| Ckb | NM_021273 | up | creatine kinase, brain |
| Clec10a | NM_010796.1 | up | C-type lectin domain family 10, |
| | | | member A |
| Cnn3 | NM_028044.1 | up | calponin 3, acidic |
| Cno | NM_133724.2 | up | cappuccino |
| Cort | NM_007745.2 | up | cortistatin |
| Cpped1 | NM_146067 | up | calcineurin-like phosphoesterase |
| | | | domain containing 1 |
| Cpsf2 | NM_016856.2 | up | cleavage and polyadenylation |
| | | | specific factor 2 |
| Ctsk | NM_007802.2 | up | cathepsin K |
| D17H6S56 | NM_033075.2 | up | DNA segment, Chr 17, human |
| E-5 | | | D6S56E 5 |
| Dab2 | NM_023118.1 | up | disabled homolog 2 (Drosophila) |
| Dcaf15 | NM_172502.2 | up | DDB1 and CUL4 associated factor |
| | | | 15 |
| Dido1 | NM_175551.2 | up | death inducer-obliterator 1 |
| Dnase1l1 | NM_027109.1 | up | deoxyribonuclease 1-like 1 |
| Emp1 | NM_010128.3 | up | epithelial membrane protein 1 |
| Erh | NM_007951.1 | up | enhancer of rudimentary homolog |
| | | | (Drosophila) |
| Erp44 | NM_029572.1 | up | endoplasmic reticulum protein 44 |
| Fabp3 | NM_010174.1 | up | fatty acid binding protein 3, muscle |
| | | | and heart |
| Fabp5 | NM_010634.1 | up | fatty acid binding protein 5, |
| | | | epidermal |
| Fam117a | NM_172543.1 | up | family with sequence similarity 117, |
| | | | member A |
| Fam125a | NM_028617.2 | up | family with sequence similarity 125, |
| | | | member A |
| Fam129b | NM_146119.1 | up | family with sequence similarity 129, |
| | | | member B |
| Fam158a | NM_033146.1 | up | family with sequence similarity 158, |
| | | | member A |
| Fam173b | NM_026546 | up | family with sequence similarity 173, |
| | | | member B |
| Fchsd2 | NM_199012.1 | up | FCH and double SH3 domains 2 |
| Fig4 | NM_133999.1 | up | FIG4 homolog (S. cerevisiae) |
| Gcnt1 | NM_010265.1 | up | glucosaminyl (N-acetyl) transferase |
| | | | 1, core 2 |
| Gdf3 | NM_008108.1 | up | growth differentiation factor 3 |
| Gmppa | NM_133708.1 | up | GDP-mannose pyrophosphorylase A |
| Golga2 | NM_133852.1 | up | golgi autoantigen, golgin subfamily a, 2 |
| Gpnmb | NM_053110.2 | up | glycoprotein (transmembrane) nmb |
| Grasp | NM_019518.2 | up | GRP1 (general receptor for |
| | | | phosphoinositides 1)-associated |
| | | | scaffold protein |
| Gtpbp2 | NM_019581.2 | up | GTP binding protein 2 |
| Gxylt1 | NM_001033275.1 | up | glucoside xylosyltransferase 1 |
| H2-K1 | NM_019909.1 | up | histocompatibility 2, K1, K region |
| H2-Q7 | NM_010394.2 | up | histocompatibility 2, Q region locus 7 |
| Haghl | NM_026897 | up | hydroxyacylglutathione hydrolase- |
| | | | like |
| Hdac10 | NM_199198.1 | up | histone deacetylase 10 |
| Hltf | NM_144959.1 | up | helicase-like transcription factor |
| Hmox1 | NM_010442.1 | up | heme oxygenase (decycling) 1 |
| Hsd3b2 | NM_153193.2 | up | hydroxy-delta-5-steroid |
| | | | dehydrogenase, 3 beta- and steroid |
| | | | delta-isomerase 2 |
| Hsd3b7 | NM_133943.2 | up | hydroxy-delta-5-steroid |
| | | | dehydrogenase, 3 beta- and steroid |
| | | | delta-isomerase 7 |
| Hspb6 | NM_001012401.1 | up | heat shock protein, alpha-crystallin- |
| | | | related, B6 |
| Hsph1 | NM_013559.1 | up | heat shock 105 kDa/110 kDa protein 1 |
| Ifih1 | NM_027835.1 | up | interferon induced with helicase C |
| | | | domain 1 |
| Ift172 | NM_026298.4 | up | intraflagellar transport 172 homolog |
| | | | (Chlamydomonas) |
| Il18r1 | NM_008365.1 | up | interleukin 18 receptor 1 |
| Irf3 | NM_016849.2 | up | interferon regulatory factor 3 |
| Isy1 | NM_133934.2 | up | ISY1 splicing factor homolog |
| | | | (S. cerevisiae) |
| Kcnab2 | NM_010598.2 | up | potassium voltage-gated channel, |
| | | | shaker-related subfamily, beta |
| | | | member 2 |
| Khnyn | NM_027143 | up | KH and NYN domain containing |
| Klhdc4 | NM_145605.1 | up | kelch domain containing 4 |
| Klra17 | NM_133203 | up | killer cell lectin-like receptor, |
| | | | subfamily A, member 17 |
| Kpna3 | NM_008466.2 | up | karyopherin (importin) alpha 3 |
| Lcmt1 | NM_025304.3 | up | leucine carboxyl methyltransferase 1 |
| Lgals1 | NM_008495.1 | up | lectin, galactose binding, soluble 1 |
| Lhfpl2 | NM_172589.1 | up | lipoma HMGIC fusion partner-like 2 |
| Lpar1 | NM_010336.1 | up | lysophosphatidic acid receptor 1 |
| Lpl | NM_008509.1 | up | lipoprotein lipase |
| Lrp12 | NM_172814.1 | up | low density lipoprotein-related |
| | | | protein 12 |
| Luc7l2 | NM_138680.1 | up | LUC7-like 2 (S. cerevisiae) |
| Ly6a | NM_010738.2 | up | lymphocyte antigen 6 complex, locus |
| | | | A |
| Ly6d | NM_010742.1 | up | lymphocyte antigen 6 complex, locus |
| | | | D |
| Mfge8 | NM_001045489.1 | up | milk fat globule-EGF factor 8 protein |
| Mrpl1 | NM_053158.1 | up | mitochondrial ribosomal protein L1 |
| Ms4a7 | NM_027836.5 | up | membrane-spanning 4-domains, |
| | | | subfamily A, member 7 |
| Mul1 | NM_026689.3 | up | mitochondrial ubiquitin ligase |
| | | | activator of NFKB 1 |
| Naglu | NM_013792.1 | up | alpha-N-acetylglucosaminidase |
| | | | (Sanfilippo disease IIIB) |
| Ndufb4 | NM_026610.1 | up | NADH dehydrogenase (ubiquinone) |
| | | | 1 beta subcomplex 4 |
| Ndufb6 | NM_001033305.1 | up | NADH dehydrogenase (ubiquinone) |
| | | | 1 beta subcomplex, 6 |
| Nelf | NM_020276.2 | up | nasal embryonic LHRH factor |
| Nol7 | NM_023554.1 | up | nucleolar protein 7 |
| Pafah1b3 | NM_008776.1 | up | platelet-activating factor |
| | | | acetylhydrolase, isoform 1b, subunit 3 |
| Pcna | NM_011045.1 | up | proliferating cell nuclear antigen |
| Pde1b | NM_008800 | up | phosphodiesterase 1B, Ca2+- |
| | | | calmodulin dependent |
| Phf11 | NM_172603.1 | up | PHD finger protein 11 |
| Pigx | NM_024464.2 | up | phosphatidylinositol glycan anchor |
| | | | biosynthesis, class X |
| Pla2g15 | NM_133792.2 | up | phospholipase A2, group XV |
| Pld3 | NM_011116.1 | up | phospholipase D family, member 3 |
| Pnpla6 | NM_015801.1 | up | patatin-like phospholipase domain |
| | | | containing 6 |
| Pold1 | NM_011131.2 | up | polymerase (DNA directed), delta 1, |
| | | | catalytic subunit |
| Pom121 | NM_148932.1 | up | nuclear pore membrane protein 121 |
| Por | NM_008898.1 | up | P450 (cytochrome) oxidoreductase |
| Pqlc2 | NM_145384 | up | PQ loop repeat containing 2 |
| Prfl | NM_011073.2 | up | perform 1 (pore forming protein) |
| Psmd8 | NM_026545.1 | up | proteasome (prosome, macropain) |
| | | | 26S subunit, non-ATPase, 8 |
| Rabep2 | NM_030566.1 | up | rabaptin, RAB GTPase binding |
| | | | effector protein 2 |
| Rbak | NM_021326.1 | up | RB-associated KRAB represser |
| Renbp | NM_023132.1 | up | renin binding protein |
| Rhbdf1 | NM_010117.1 | up | rhomboid family 1 (Drosophila) |
| Robld3 | NM_031248.3 | up | roadblock domain containing 3 |
| Sbf1 | NM_001081030.1 | up | SET binding factor 1 |
| Sdc3 | NM_011520.2 | up | syndecan 3 |
| Sec11a | NM_019951.1 | up | SEC11 homolog A (S. cerevisiae) |
| Serpinb6a | NM_009254 | up | serine (or cysteine) peptidase |
| | | | inhibitor, clade B, member 6a |
| Sfxn4 | NM_053198 | up | sideroflexin 4 |
| Sh3pxd2b | NM_177364 | up | SH3 and PX domains 2B |
| Siglec1 | NM_011426.1 | up | sialic acid binding Ig-like lectin 1, |
| | | | sialoadhesin |
| Slamf6 | NM_030710 | up | SLAM family member 6 |
| Slc23a2 | NM_018824.2 | up | solute carrier family 23 (nucleobase |
| | | | transporters), member 2 |
| Slc25a10 | NM_013770 | up | solute carrier family 25 |
| | | | (mitochondrial carrier, dicarboxylate |
| | | | transporter), member 10 |
| Slc35e3 | NM_029875 | up | solute carrier family 35, member E3 |
| Slc36a1 | NM_153139.3 | up | solute carrier family 36 |
| | | | (proton/amino acid symporter), |
| | | | member 1 |
| Slc5a6 | NM_177870.2 | up | solute carrier family 5 (sodium- |
| | | | dependent vitamin transporter), |
| | | | member 6 |
| Slc6a8 | NM_133987.1 | up | solute carrier family 6 |
| | | | (neurotransmitter transporter, |
| | | | creatine), member 8 |
| Slc9a7 | NM_177353.2 | up | solute carrier family 9 |
| | | | (sodium/hydrogen exchanger), |
| | | | member 7 |
| Snx1 | NM_019727.1 | up | sorting nexin 1 |
| Snx11 | NM_028965.2 | up | sorting nexin 11 |
| Spns1 | NM_023712.1 | up | spinster homolog 1 (Drosophila) |
| Srp14 | NM_009273.2 | up | signal recognition particle 14 |
| Srsf7 | NM_146083.1 | up | serine/arginine-rich splicing factor 7 |
| Sspn | NM_010656.1 | up | sarcospan |
| Ssr4 | NM_009279 | up | signal sequence receptor, delta |
| Stat6 | NM_009284.2 | up | signal transducer and activator of |
| | | | transcription 6 |
| Tap2 | NM_011530.2 | up | transporter 2, ATP-binding cassette, |
| | | | sub-family B (MDR/TAP) |
| Tbrg1 | NM_025289.1 | up | transforming growth factor beta |
| | | | regulated gene 1 |
| Tchp | NM_029992.1 | up | trichoplein, keratin filament binding |
| Tcta | NM_133986 | up | T-cell leukemia translocation altered |
| | | | gene |
| Tmem106a | NM_144830.1 | up | transmembrane protein 106A |
| Tmem51 | NM_145402.2 | up | transmembrane protein 51 |
| Tmem65 | NM_175212.4 | up | transmembrane protein 65 |
| Tnfrsf26 | NM_175649.2 | up | tumor necrosis factor receptor |
| | | | superfamily, member 26 |
| Tpcn2 | NM_146206 | up | two pore segment channel 2 |
| Trem2 | NM_031254.2 | up | triggering receptor expressed on |
| | | | myeloid cells 2 |
| Tsc2 | NM_001039363.1 | up | tuberous sclerosis 2 |
| Tsc22d3 | NM_001077364.1 | up | TSC22 domain family, member 3 |
| Tspan32 | NM_020286.2 | up | tetraspanin 32 |
| Ube2q1 | NM_027315.2 | up | ubiquitin-conjugating enzyme E2Q |
| | | | (putative) 1 |
| Unc45a | NM_133952.1 | up | unc-45 homolog A (C. elegans) |
| Vpreb3 | NM_009514.2 | up | pre-B lymphocyte gene 3 |
| Zbtb22 | NM_020625.2 | up | zinc finger and BTB domain |
| | | | containing 22 |
| Zfhx2 | NM_001039198.1 | up | zinc finger homeobox 2 |
| Zfp467 | NM_020589.1 | up | zinc finger protein 467 |
| Zfp787 | NM_001013012.1 | up | zinc finger protein 787 |
| Zgpat | NM_144894.2 | up | zinc finger, CCCH-type with G patch |
| | | | domain |
| Zxda | NR_003292.1 | up | zinc finger, X-linked, duplicated A |

### Example 7

### dhS1P Abrogates the Propagation of Tumor-Amplified Immature Myeloid Cells that Allows Concomitant Expansion of Antitumor Lymphocytes

According to a specific aspect of the invention, the effects of dhS1P at the level of hematopoietic cells were evaluated. Specifically, the effects of dhS1P were directly compared with those of S1P as to delineate a difference in their physiological roles. Tumor-expanded immature myeloid cells were isolated and exposed in culture to either dhS1P or S1P. Given the robust increase in dhS1P compared with S1P that was observed in response to PhotoImmunoNanoTherapy in the in vivo studies, it was of little surprise that only dhS1P exerted an effect on isolated immature myeloid cells in culture. Specifically, multicolor flow cytometry revealed that cells bearing the surface characteristics of immature myeloid cells were completely ablated under normal culture conditions by dhS1P treatment but not S1P treatment (FIG. 7A). This was confirmed by repeating the same dhS1P, or S1P, treatments on isolated immature myeloid cells but in growth-factor-supplemented media as a colony-forming assay. Isolated immature myeloid cells were cultured in CFU (colony-forming unit)-GEMM (granulocyte, erythrocyte, monocyte, megakaryocyte) supportive semisolid media and formed GEMM colonies indicative of their multipotent myeloid progenitor nature (FIG. 7B). This specific colony growth was shown to be dramatically augmented by S1P treatment. In contrast, CFU-GEMM colony formation was completely abrogated by exposure to dhS1P, indicative of the lipid's potent regulatory effect. Intriguingly, dhS1P exposure also promoted the expansion of a new population of cells in culture which displayed CD19 and CD45R B220 on their surface—markers that are indicative of B-cells (FIG. 7A). Importantly, we observed this same expansion of CD19+CD45R B220+ cells within splenocyte isolations from tumor-bearing mice treated with PhotoImmunoNanoTherapy. In addition, PhotoImmunoNanoTherapy triggered the expansion of cells bearing the expression of the natural killer (NK) cell marker CD49b DX5—a lymphocyte population known for antitumor activity. It is possible that these effects are indirect, in that dhS1P-mediated suppression of immature myeloid cells simply removes a blockade of lymphoid differentiation. In agreement with this idea, we observed that dhS1P mediated the expansion of the same CD19+CD45R B220+ cellular population from isolated hematopoietic progenitors (FIG. 7C). The inventors separately performed lineage tracing analysis to confirm that this population is not of myeloid origin (FIG. 8).

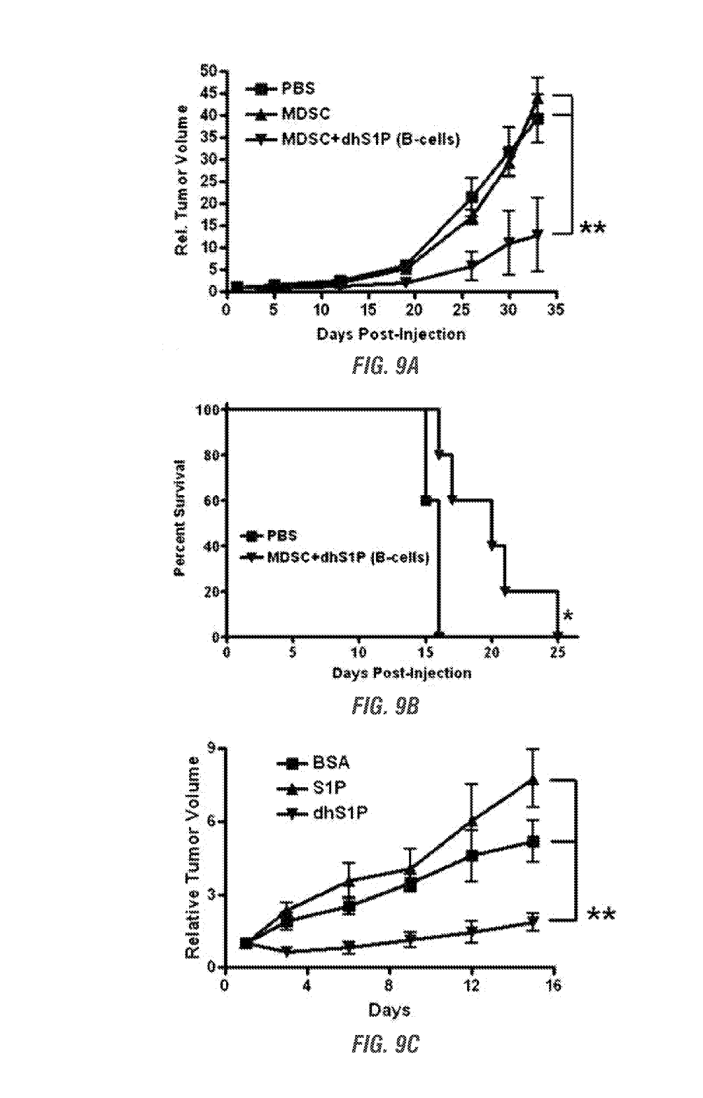

Collectively, the above examples show that dhS1P, a product of PhotoImmunoNanoTherapy-stimulated SphK activity, can negatively regulate IMCs that are expanded as part of the tumor-associated pro-inflammatory milieu, which indirectly promotes the expansion of other lymphoid-origin cells. These lymphoid-origin cells were further isolated, which bear the surface characteristics of B-cells, and adoptively transferred them into breast cancer and pancreatic cancer-bearing hosts to achieve therapeutic responses evidenced respectively by decreased breast cancer tumor growth or an extension of survival in a model bearing orthotopic pancreatic cancer (FIG. 9A-B). Separately, tumor-bearing mice were injected with dhS1P and observed a therapeutic effect (FIG. 9C). As expected, injection of S1P in this same experiment resulted in augmented tumor growth, owing to the well-defined role of S1P in tumor growth and progression (FIG. 9C). Altogether, these results showed that dhS1P could mediate the development of an antitumor lymphocyte population. These experiments also offer confirmation that the increase in dhS1P observed in response to PhotoImmunoNanoTherapy is responsible for its immunoregulatory and antitumor effects.

### Example 8

### Materials and Methods

Reagents.

Cell culture media was purchased from Mediatech (Manassas, Va.), FBS was obtained from Gemini Bio-Products (West Sacramento, Calif.), and other cell culture reagents were from Invitrogen (Carlsbad, Calif.). Antibodies were from eBiosciences (San Diego, Calif.), BD Biosciences (San Jose, Calif.), Miltenyi Biotech (Bergisch Gladbach, Germany), and Santa Cruz Biotechnology (Santa Cruz, Calif.). Unless specified else wise, other reagents were from Sigma (St. Louis, Mo.).

Cell Culture.

Human BxPC-3 cells were cultured in RPMI-1640 supplemented with 10% FBS and antibiotic-antimycotic solution. Human MDA-MB-231 cells, human SAOS-2-LM7 cells, murine 410.4 cells, and murine Panc-02 cells, were cultured in DMEM supplemented with 10% FBS and antibiotic-antimycotic solution. All cultures were maintained at 37° C. and 5% CO2.

CPSNP Preparation.

PEGylated CPSNPs loaded with ICG were prepared as previously described (6-10). Briefly, a water-in-oil microemulsion using a cyclohexane/Igepal C-520/water system was used to self-assemble reverse micelles that served as templates for the size controlled precipitation, and surface functionalization, of the nanoparticles. Calcium and phosphate, with metasilicate doping, were used as the matrix materials with entrapment of the ICG achieved by matrix precipitation around the fluorophore molecules confined within a reverse micelle. Citrate functionalization was achieved by specific adsorption, providing carboxylate groups for secondary PEG functionalization. A van der Waals laundering procedure was used to remove spectator ions, amphiphiles, and the hydrophobic phase. 1-ethyl-3-(3-dimethylaminopropyl) carbodiimide was used to conjugate methoxy-terminated PEG to the CPSNPs. Lastly, centrifugation was used to further wash and concentrate the CPSNPs.

Animal Trials.

Orthotopic pancreatic cancer and subcutaneous breast cancer tumors were established in athymic nude, NOD.CB17-Prkdc^{scid}/J, BALB/cJ, or C57BL/6J mice as previously described (8, 9), with minor modifications. All cell lines used in animal and cellular studies, prior to any modification, were originally obtained from the American Type Culture Collection (Manassas, Va.). For orthotopic BxPC-3-GFP human pancreatic cancer xenografts, 4-6 week old female athymic mice were fully anesthetized with a mixture of ketamine-HCl (129 mg/kg) and xylazine (4 mg/kg) injected intramuscularly. A small incision was made in the left flank, the peritoneum was dissected and the pancreas exposed. Using a 27-gauge needle, 2.5×10⁶ cells, prepared in 0.1 mL of Hank's balanced salt solution, were injected into the pancreas. For experimental lung-metastatic osteosarcoma xenografts, 4-6 week old female athymic nude mice were tail vein-injected with 2.5×10⁶ human SAOS-2-LM7 cells. For a subcutaneous MDA-MB-231 human breast cancer model, 1×10⁷ cells were prepared in 0.2 mL of normal growth media, and injected subcutaneously, on each side, into 4-6 week old female athymic nude mice. For subcutaneous 410.4 murine breast cancer models, 2.5×10⁵ cells were similarly prepared and injected into 7 week old female BALB/cJ or 5 week old female NOD.CB17-Prkdc^{scid}/J mice. For a subcutaneous Panc-02 murine pancreatic cancer model, 2×10⁶ cells were prepared in 0.2 mL of normal growth media, and injected subcutaneously, on each side, into 7 week old male C57BL/6J mice. All tumor models were allowed to establish for at least one week prior to experimentation. For PhotoImmunoNanoTherapy, tumor-bearing mice weighing approximately 20 grams received 0.1 mL injections of ICG-CPSNPs diluted approximately 1:10 into PBS (200 nM pre-injection concentration of ICG), or controls, followed 24 hours later by 12.5 J/cm² laser NIR irradiation of the subcutaneous tumors, the pancreas, or the lungs (one injection for the MDA-MB-231 breast cancer model, every third day injections for other subcutaneous cancer models, three weekly injections for the orthotopic pancreatic cancer model, and five weekly injections for the metastatic osteosarcoma model). For studies evaluating knockdown of sphingosine kinase, siRNA-transfected MDA-MB-231 cells treated first in culture with PhotoImmunoNanoTherapy were tail-vein injected into tumor-bearing mice (note, for this trial the initial tumor sizes were larger to allow for less growth-related variation). Tumor size was measured by caliper measurement. For adoptive transfer studies, IMCs isolated from splenocytes were treated in culture with sphingolipids prior to adoptive transfer into breast- or pancreatic tumor-bearing athymic nude mice. For studies evaluating the specific tumor-modulating effects of phosphorylated bioactive sphingolipids, C57BL/6J mice engrafted with subcutaneous Panc-02 pancreatic cancer tumors were injected every other day with sphingolipids conjugated to a BSA carrier protein (0.1 mL of an initial concentration of 100 μM). Survival to pre-determined humane endpoints was monitored for some studies. In other studies, mice were sacrificed following NIR laser treatment for tumor or serum analysis. All animal procedures were approved by the Pennsylvania State University College of Medicine Institutional Animal Care and Use Committee.

Cell Sorting and Flow Cytometry.

Splenocytes were harvested from tumor-bearing mice by mechanical disruption in red blood cell lysis buffer. Splenocytes were washed, and resuspended in PBS with Mouse BD Fc Block (1 μg per 1×10⁶ splenocytes), and incubated for 15 minutes at 4° C. For IMC isolation, antibodies targeting Gr-1 (FITC) and CD11b (PE-Cy7) were added. Splenocytes were incubated for 15 minutes at 4° C. with the respective antibodies (1 μg per 1×10⁶ splenocytes). Cell isolation was performed by the Pennsylvania State University College of Medicine Flow Cytometry Core Facility utilizing a Dako Cytomation MoFlo High Performance cell sorter (purity 85-95%) For flow cytometry, splenocytes were prepared in similar fashion with antibodies targeting Gr-1 (FITC, or APC-eFluor 780), CD11b (PE-Cy7), CD44 (eFluor 605NC), CD115 (PE), gp91^{phox} (DyLight 649), or LY-6C (PerCP-Cy5.5). Multicolor flow cytometry was performed at the Pennsylvania State University College of Medicine Flow Cytometry Core Facility utilizing a BD Biosciences LSR II Special Order flow cytometer. BD FACS Diva software was used to analyze results. All antibodies were purchased from eBioscience, BD Biosciences, or Santa Cruz. DyLight conjugations were performed with a conjugation kit from Thermo Fisher.

CFU-GEMM Assay.

Isolated IMCs from the spleens of tumor-bearing athymic nude mice were cultured (5×10⁴ cells/mL) in GEMM-supportive complete (mouse) methylcellulose media (R&D Systems, Minneapolis, Minn.), according to the manufacturer's instructions, with BSA, S1P (5 μM), or dhS1P (5 μM). GEMM colonies were visualized and counted after 3 weeks of culture.

Lipidomics.

Lipids were extracted from tumors or serum using a modified Bligh-Dyer extraction. Extracts were subjected to liquid chromatography and electrospray ionoization-tandem mass spectroscopy (LC-ESI-MS³) to detect sphingolipid metabolites, as previously described (28).

Cytokine Multiplex Assay.

An R&D Systems Fluorokine MultiAnalyte Profiling kit was used according to the manufacturer's instructions. Briefly, serum was diluted 1:4 into calibrator diluent RD6-40 and then added to a microplate containing analyte-specific microparticles. A biotin antibody cocktail and streptavidin-PE were added according to the manufacturer's instructions, including wash and incubation steps. Lastly, the mixtures were resuspended in wash buffer and analyzed using a BioRad BioPlex analyzer.

RNA Interference.

MDA-MB-231 cells were subcultured and allowed to grow until 50-60% confluent. SphK1 (Dharmacon catalog number: M-004172-03; accession number: NM_{—}021972), SphK2 (Dharmacon catalog number: M-004831-00; accession number: NM_{—}020126), or non-targeted pools of siRNA (Dharmacon catalog number: D-001206-14, Pool #2), were transfected with Lipofectamine 2000 according to the manufacturer's instructions. Cells were harvested 24 hours post-transfection.

Statistics. GraphPad Prism 5.0 software was used to plot graphs as well as to determine significance of results. ANOVA (1-way or 2-way), followed by Bonferroni comparisons, or an unpaired student's t-test, were used to determine significance between treatment groups. A logrank test was used to determine significance of survival between treatment groups. All data represent averages±standard error of the mean.

MicroArray. Isolated MDSC-like cells were cultured for 24 hours in media containing BSA, or dhS1P (5 μM), before collection and washing via centrifugation. RNA was extracted, and microarray analysis was performed by the Pennsylvania State University College of Medicine Functional Genomics Core Facility utilizing Illumina technology (Illumina, San Diego, Calif.), according to standard procedures. For RNA amplification, the Illumina TotalPrep RNA Amplification kit was used standard procedures. Briefly, 50-100 ng of RNA was reverse transcribed to synthesize first strand cDNA by incubating samples at 42° C. for 2 hours with T7 Oligo(dT) primer, 10× first strand buffer, dNTPs, RNAse inhibitor, and ArrayScript. Second strand cDNA was synthesized with 10× second strand buffer, dNTPs, DNA polymerase and Rnase H at 16° C. for 2 hours. cDNA was purified according to standard procedures. cDNA was in vitro transcribed to synthesize cRNA using a MEGAscript kit (Ambion, Austin, Tex.). Samples were incubated with T7 10× reaction buffer, T7 Enzyme mix and Biotin-NTP mix at 37° C. for 14 hours. cRNA was purified according to instructions, and the yield was measured using a NanoDrop ND-1000 (NanoDrop Products, Wilmington, Del.). 750 ng of purified cRNA was prepared for hybridization according to instructions for hybridizing to Illumina MouseRef-8 Expression BeadChips. BeadChips were incubated in a hybridization oven for 20 hours at 58° C. at a rocker speed of 5. After 20 hours, BeadChips were disassembled, washed, and Streptavadin-Cy3 stained according to Illumina standard procedures. BeadChips were dried by centrifugation at 275×g for 4 minutes and subsequently scanned using a BeadArray Reader.

Data was imported into GeneSpring GX 7.3 (Agilent Technologies, Santa Clara, Calif.) and signal values less than 0.01 were set to 0.01, and individual genes normalized to the median. Values were then normalized on a per gene basis to the BSA-treated group. Potential differential gene expression was determined with a one-way ANOVA, p<0.05 and filtered for 1.2 fold or greater differences in expression in accordance with standards for microarray analysis. Ingenuity Pathway Analysis (Ingenuity Systems, Redwood City, Calif.) was used to evaluate pathways and networks of genes that were shown to be differentially expressed.

### REFERENCES

The following references are referred to in the specification and are incorporated by reference as if set forth fully herein:
1. Ortel, B.; Shea, C. R.; Calzavara-Pinton, P. Molecular mechanisms of photodynamic therapy. Front. Biosci. 2009, 14, 4157-4172.
2. Juarranz, A.; Jaen, P.; Sanz-Rodriguez, F.; Cuevas, J.; Gonzalez, S. Photodynamic therapy of cancer. basic principles and applications. Clin. Trans'. Oncol. 2008, 10, 148-154.
3. Almeida, R. D.; Manadas, B. J.; Carvalho, A. P.; Duarte, C. B. Intracellular signaling mechanisms in photodynamic therapy. Biochim. Biophys. Acta. 2004, 1704, 59-86.
4. Wainwright, M. Photodynamic therapy: The development of new photosensitisers. Anticancer Agents Med. Chem. 2008, 8, 280-291.
5. Chatterjee, D. K.; Fong, L. S.; Zhang, Y. Nanoparticles in photodynamic therapy: An emerging paradigm. Adv. Drug Deliv. Rev. 2008, 60, 1627-1637.
6. Morgan, T. T.; Muddana, H. S.; Altino\check{g}lu, E. I.; Rouse, S. M.; Tabaković, A.; Tabouillot, T.; Russin, T. J.; Shanmugavelandy, S. S.; Butler, P. J.; Eklund, P. C.; et al. Encapsulation of organic molecules in calcium phosphate nanocomposite particles for intracellular imaging and drug delivery. Nano Lett. 2008, 8, 4108-4115.
7. Kester, M.; Heakal, Y.; Fox, T.; Sharma, A.; Robertson, G. P.; Morgan, T. T.; Altino\check{g}lu, E. I.; Tabaković, A.; Parette, M. R.; Rouse, S. M.; et al. Calcium phosphate nanocomposite particles for in vitro imaging and encapsulated chemotherapeutic drug delivery to cancer cells. Nano Lett. 2008, 8, 4116-4121.
8. Altino\check{g}lu, E. I.; Russin, T. J.; Kaiser, J. M.; Barth, B. M.; Eklund, P. C.; Kester, M.; Adair, J. H. Near-infrared emitting fluorophore-doped calcium phosphate nanoparticles for in vivo imaging of human breast cancer. ACS Nano 2008, 2, 2075-2084.
9. Barth, B. M.; Sharma, R.; Altino\check{g}lu, E. I.; Morgan, T. T.; Shanmugavelandy, S. S.; Kaiser, J. M.; McGovern, C.; Matters, G. L.; Smith, J. P.; Kester, M.; et al. Bioconjugation of calcium phosphosilicate composite nanoparticles for selective targeting of human breast and pancreatic cancers in vivo. ACS Nano 2010, 4, 1279-1287.
10. Muddana, H. S.; Morgan, T. T.; Adair, J. H.; Butler, P. J. Photophysics of Cy3-encapsulated calcium phosphate nanoparticles. Nano Lett. 2009, 9, 1559-1566.
11. Barth, B. M.; Altino\check{g}lu, E. I.; Shanmugavelandy, S. S.; Kaiser, J. M.; Crespo-Gonzalez, D.; DiVittore, N. A.; McGovern, C.; Goff, T. M.; Keasey, N. R.; Adair, J. H.; et al. Targeted indocyanine green-loaded calcium phosphosilicate nanoparticles for in vivo photodynamic therapy of leukemia. ACS Nano 2011, 5, 5325-5337.
12. Separovic, D.; Bielawski, J.; Pierce, J. S.; Merchant, S.; Tarca, A. L.; Ogretmen, B.; Korbelik, M. Increased tumour dihydroceramide production after photofrin-PDT alone and improved tumour response after the combination with the ceramide analogue LCL29. evidence from mouse squamous cell carcinomas. Br. J. Cancer 2009, 100, 626-632.
13. Saddoughi, S. A.; Song, P.; Ogretmen, B. Roles of bioactive sphingolipids in cancer biology and therapeutics. Subcell. Biochem. 2008, 49, 413-440.
14. Gouaze-Andersson, V.; Yu, J. Y.; Kreitenberg, A. J.; Bielawska, A.; Giuliano, A. E.; Cabot, M. C. Ceramide and glucosylceramide upregulate expression of the multidrug resistance gene MDR1 in cancer cells. Biochim. Biophys. Acta. 2007, 1771, 1407-1417.
15. Ogretmen, B. Sphingolipids in cancer: Regulation of pathogenesis and therapy. FEBS Lett. 2006, 580, 5467-5476.
16. Hannun, Y. A.; Obeid, L. M. Principles of bioactive lipid signalling: Lessons from sphingolipids. Nat. Rev. Mol. Cell. Biol. 2008, 9, 139-150.
17. Lahiri, S.; Futerman, A. H. The metabolism and function of sphingolipids and glycosphingolipids. Cell. Mol. Life. Sci. 2007, 64, 2270-2284.
18. Maceyka, M.; Milstien, S.; Spiegel, S. Sphingosine-1-phosphate: The swiss army knife of sphingolipid signaling. J. Lipid Res. 2009, 50 Suppl, S272-S276.
19. Oskeritzian, C. A.; Price, M. M.; Hait, N.C.; Kapitonov, D.; Falanga, Y. T.; Morales, J. K.; Ryan, J. J.; Milstien, S.; Spiegel, S. Essential roles of sphingosine-1-phosphate receptor 2 in human mast cell activation, anaphylaxis, and pulmonary edema. J. Exp. Med. 2010, 207, 465-474.
20. Davis, M. D.; Kehrl, J. H. The influence of sphingosine-1-phosphate receptor signaling on lymphocyte trafficking. How a bioactive lipid mediator grew up from an “immature” vascular maturation factor to a “mature” mediator of lymphocyte behavior and function. Immunol. Res. 2009, 43, 187-197.
21. Rivera, J.; Proia, R. L.; Olivera, A. The alliance of sphingosine-1-phosphate and its receptors in immunity. Nat. Rev. Immunol. 2008, 8, 753-763.
22. Ostrand-Rosenberg, S.; Sinha, P. Myeloid-derived suppressor cells: Linking inflammation and cancer. J. Immunol. 2009, 182, 4499-4506.
23. Gabrilovich, D. I.; Nagaraj, S. Myeloid-derived suppressor cells as regulators of the immune system. Nat. Rev. Immunol. 2009, 9, 162-174.
24. Corzo, C. A.; Cotter, M. J.; Cheng, P.; Cheng, F.; Kusmartsev, S.; Sotomayor, E.; Padhya, T.; McCaffrey, T. V.; McCaffrey, J. C.; Gabrilovich, D. I. Mechanism regulating reactive oxygen species in tumor-induced myeloid-derived suppressor cells.J. Immunol. 2009, 182, 5693-5701.
25. Li, H.; Han, Y.; Guo, Q.; Zhang, M.; Cao, X. Cancer-expanded myeloid-derived suppressor cells induce anergy of NK cells through membrane-bound TGF-beta 1. J. Immunol. 2009, 182, 240-249.
26. Cheng, P.; Corzo, C. A.; Luetteke, N.; Yu, B.; Nagaraj, S.; Bui, M. M.; Ortiz, M.; Nacken, W.; Sorg, C.; Vogl, T.; et al Inhibition of dendritic cell differentiation and accumulation of myeloid-derived suppressor cells in cancer is regulated by S100A9 protein. J. Exp. Med. 2008, 205, 2235-2249.
27. Youn, J. I.; Nagaraj, S.; Collazo, M.; Gabrilovich, D. I. Subsets of myeloid-derived suppressor cells in tumor-bearing mice. J. Immunol. 2008, 181, 5791-5802.
28. Wijesinghe, D. S.; Allegood, J. C.; Gentile, L. B.; Fox, T. E.; Kester, M.; Chalfant, C. E. Use of high performance liquid chromatography-electrospray ionization-tandem mass spectrometry for the analysis of ceramide-1-phosphate levels. J. Lipid Res. 2010, 51, 641-651.
29. Hait, N.C.; Allegood, J.; Maceyka, M.; Strub, G. M.; Harikumar, K. B.; Singh, S. K.; Luo, C.; Marmorstein, R.; Kordula, T.; Milstien, S.; et al. Regulation of histone acetylation in the nucleus by sphingosine-1-phosphate. Science 2009, 325, 1254-1257.
30. Gross, S. A.; Wolfsen, H. C. The role of photodynamic therapy in the esophagus. Gastrointest. Endosc. Clin. N. Am. 2010, 20, 35-53, vi.
31. Kotimaki, J. Photodynamic therapy of eyelid basal cell carcinoma. J. Eur. Acad. Dermatol. Venereol. 2009, 23, 1083-1087.
32. Cuvillier, O. Downregulating sphingosine kinase-1 for cancer therapy. Expert Opin. Ther. Targets 2008, 12, 1009-1020.

All patents, patent applications, publications, and descriptions mentioned throughout the specification are herein incorporated by reference in their entirety for all purposes. None is admitted to be prior art.

The invention has been described with reference to various specific and preferred embodiments and techniques. However, it should be understood that many variations and modifications may be made while remaining within the spirit and scope of the invention.

What is claimed is:

1. A method of modulating the immune system of a patient in need thereof comprising: administering an effective amount of dhS1P to the subject, wherein said dshS1P decreases the number of MDSCs in said subject.

2. The method of claim 1 wherein the dhS1P is encapsulated in calcium phosphosilicate nanoparticles.

3. The method of claim 1, wherein the route of said administering is topical, intravenous, oral, subcutaneous, local, subcutaneous, intramuscular, or by use of an implant.

4. A composition for treating cancer, comprising dihydrosphingosine-1-phosphate (dhS1P) and a pharmaceutically-acceptable carrier.

5. The composition of claim 4 wherein the composition further comprises a cancer therapy agent.

6. The composition of claim 5 wherein the cancer therapy agent is a cancer immunotherapy agent.

7. The composition of claim 4 wherein said composition is formulated for topical, intravenous, oral, subcutaneous, local, subcutaneous, or intramuscular administration or administration by use of an implant.

8. The composition of claim 4 wherein said dhS1P is encapsulated in calcium phosphosilicate nanoparticles.

9. A method of adjuvant, neoadjuvant or concomitant cancer therapy comprising administering to a host that has or is suspected to have a cancer, an effective amount of dhS1P and at least one additional cancer treatment.

10. The method of claim 9 wherein the additional cancer therapy treatement is administration of a cancer therapy agent.

11. The method of claim 10 wherein the cancer therapy agent is a cancer immunotherapy agent.

12. The method of claim 9, wherein said cancer is a cancer wherein elevated levels of MDSCs are observed.

13. The method of claim 9, wherein said cancer is pancreatic cancer, lung-metastatic osteosarcoma, or breast cancer.

14. The method of claim 9 wherein the number of MDSCs in said host is decreased following said administering an effective amount of dhS1P and at least one additional cancer treatment.

15. A method of adjuvant, neoadjuvant or concomitant cancer therapy comprising:
a) exposing a plurality of cancer cells to dhS1P,
b) harvesting said cells, and
c) administering said cells to a patient in need of cancer therapy.

16. The method of claim 15 wherein the exposure to dhS1P is accomplished through use of PhotoImmunoNanoTherapy.

17. The method of claim 16 wherein said use of PhotoImmunoNanoTherapy comprises encapsulating dhSP1 in calcium phosphosilicate nanoparticles.

18. The method of claim 16 wherein said exposure to dhS1P accomplished through use of PhotoImmunoNano Therapy comprises inducing an increase of endogenous dhS1P.

19. A method for creating a cancer therapeutic comprising:
a) exposing a plurality of cancer cells, IMCs/MDSCs, or hematopoietic progenitors to dhS1P,
b) harvesting said cells, and
c) packaging said cells.

20. A cancer therapeutic made by the method of claim 19.

21. The method of claim 19 wherein the exposure to dhS1P is accomplished through use of PhotoImmunoNanoTherapy.
